(19) United States
(12) Patent Application Publication
(10) Pub. No.: US 2019/0079078 A1
(43) Pub. Date: Mar. 14, 2019

(54) DEVICES, COMPOSITIONS AND RELATED METHODS FOR DIAGNOSING AUTISM

(71) Applicant: The Regents of the University of California, Oakland, CA (US)

(72) Inventors: Jay Gargus, Irvine, CA (US); Galina Schmunk, Anaheim, CA (US); Ian Parker, Irvine, CA (US); Ian Smith, Corona Del Mar, CA (US)

(73) Assignee: The Regents of the University of California, Oakland, CA (US)

(21) Appl. No.: 15/750,492

(22) PCT Filed: Aug. 5, 2016
(86) PCT No.: PCT/US16/45881

§ 371 Date: Feb. 5, 2018

Related U.S. Application Data
(63) Continuation-in-part of application No. 14/821,555, filed on Aug. 7, 2015.
(60) Provisional application No. 62/219,085, filed on Sep. 15, 2015.

(51) Int. Cl. G01N 33/50; G01N 33/68; C12N 5/077

(52) U.S. Cl. CPC G01N 33/5058; G01N 2800/28; C12N 5/0656; G01N 33/6872

(57) ABSTRACT

Disclosed herein are biomarkers for determining susceptibility to Autism Spectrum Disorder (ASD). Susceptibility to ASD is determined by detecting IP3R Ca2+ signaling activity level in cells, wherein a decrease in IP3R Ca2+ activity is indicative of ASD susceptibility. Also disclosed herein are methods of screening a therapeutic agent for ASD. A candidate drug is determined to be a therapeutic agent for treatment of ASD if the IP3R Ca2+ signaling activity is higher in the presence of the candidate drug than in its absence. Further disclosed herein are methods for prognosis, diagnosis, or treatment for an ASD, comprising determining IP3R Ca2+ signaling activity level in a said biological sample; and comparing it to a reference value from a control subject, where a lower activity level than the reference value in the sample is indicative of the presence of an autism spectrum disorder.

### FIELD OF THE INVENTION

The present disclosure is in the medical and biomedical field, specifically as it relates to autism.

### BACKGROUND OF THE DISCLOSURE

All publications herein are incorporated by reference to the same extent as if each individual publication or patent application was specifically and individually indicated to be incorporated by reference. The following description includes information that may be useful in understanding the present invention. It is not an admission that any of the information provided herein is prior art, or that any publication specifically or implicitly referenced is prior art.

Autism spectrum disorder (ASD) is a neurological disorder characterized by signs and symptoms that include lack of social skills, language deficiency, and stereotypic repetitive behaviors. Each of the expressivity and severity of ASD symptoms is highly variable from patient to patient; and the etiology of ASD is ill defined. However, its high heritability suggests a strong genetic component; and it is generally understood that ASD can manifest from both monogenic and polygenic disorders.

Monogenic causes of ASD are responsible for only a few percent of all cases. Still, monogenic ASD models provide tractable systems for identifying and studying the molecular mechanisms and genetic architectures that underlie ASD. Fragile X syndrome (FXS) is the most common monogenic cause of ASD, and one of the most widely used and characterized ASD models. FXS is caused by a pathogenic expansion of a CGG repeat on the X chromosome, leading to transcriptional silencing of the fragile X mental retardation (FMR1) gene. The fragile X mental retardation protein (FMRP) normally binds to several mRNAs, regulating their translation. The loss of FMRP in FXS patients leads to substantial cognitive impairment and intracellular signaling defects, both in humans and in mice. FMR1 knockout mouse lines are available and amount to tractable animal models for ASD.

Tuberous sclerosis (TS) is another monogenic cause of ADS. It is caused by dominant mutations in one of two genes, TSC1 or TSC2, which code for the proteins hamartin and tuberin, respectively. Hamartin and tuberin proteins form a functional signaling complex; and the disruption of these genes in the brain results in abnormal cellular differentiation, migration, and proliferation. TSC1 and TSC2 knockout mice are also available and amount to tractable animal models for ASD.

At present, there are no objective biomarkers of the disorder. As such diagnosis of ASD is strictly clinical. Thus there remains a need in the field for a laboratory diagnosis of ASD.

### SUMMARY OF THE INVENTION

In one embodiment, disclosed herein are kits for determining susceptibility to ASD, comprising an assay for determining an increase or decrease of IP₃R Ca²⁺ signaling activity level in cells, wherein a decrease in IP₃R Ca²⁺ activity is indicative of ASD susceptibility. In one embodiment, the IP₃R Ca²⁺ signaling activity is determined by using one or more biomarkers that is a clinically tractable discriminant of ASD. In one embodiment, the ASD is monogenic ASD or sporadic ASD. In one embodiment, the monogenic form of ASD comprises FXS, TSC1, and/or TSC2. In one embodiment, the cells comprise fibroblast cells or neuronal cells. In one embodiment, the decrease of IP₃R Ca²⁺ signaling activity arises at the IP3R channel, without a mutation in the IP₃R. In one embodiment, IP₃R Ca²⁺ signaling activity is measured by imaging Ca²⁺ flux though single ion channels within intact cells with single channel resolution. In one embodiment, IP₃R Ca²⁺ signaling activity is measured by the following: using total internal reflection microscopy together with a slow Ca²⁺ buffer to restrict excitation of a cytosolic fluorescent Ca²⁺ indicator to within ˜100 nm of the plasma membrane; monitoring the local microdomain of elevated cytosolic [Ca²⁺] around the pore of Ca²⁺-permeable membrane channels; and dissecting the Ca²⁺ puffs arising from clusters of IP₃Rs by using localized single-channel Ca²⁺ fluorescence transients, wherein the single-channel Ca²⁺ fluorescence transients turn on and off rapidly, tracking channel openings and closings with a time resolution of a few ms. In one embodiment, a change in the Ca²⁺ signaling activity is determined through changes in the spatial distribution of IP₃R channels as imaged by super-resolution imaging. In one embodiment, IP₃R Ca²⁺ signaling activity is determined by an assay comprising monitoring cytosolic Ca²⁺ signals in skin fibroblasts from FXS and matched control subjects using a fluorimetric imaging plate reader; applying ATP to activate GPCR-linked purinergic P2Y receptors in Ca²⁺ free extracellular solution to exclude Ca²⁺ influx through plasmalemmel channels; and determining changes in IP₃R Ca²⁺ signaling activity. In one embodiment, identifying the reduced IP₃R Ca²⁺ signaling activity level further comprises obtaining equivalent amounts of separately cultured skin fibroblast cells from the patient and from the control individual, wherein the cultured skin fibroblast cells from each of the patient and the control individual have been loaded with a Ca²⁺ fluorescent probe, and contacted with an agonist of IP₃R Ca²⁺ signaling; measuring, in each of the cultured fibroblast cells from the patient and the individual obtained in (a), an amount of fluorescence emitted by the Ca²⁺ fluorescent probe; and comparing the amounts of emitted fluorescence measured in (b).

In another embodiment, disclosed herein is a method of screening a therapeutic agent for ASD comprising providing a cell sample of a subject diagnosed with ASD; detecting the IP₃R Ca²⁺ signaling activity in the cell sample in the presence, as well as the absence of a candidate drug; and determining that the candidate drug is a therapeutic agent for treatment of ASD if the IP₃R Ca²⁺ signaling activity is higher in the presence of the candidate drug than in its absence. In one embodiment, the ASD is monogenic ASD or sporadic ASD. In one embodiment, the monogenic form of ASD comprises FXS, TSC1, and/or TSC2. In one embodiment, the cell sample comprises a fibroblast cell sample, an amniocyte cell sample obtained prenatally by amniocentesis, or a neuronal cell sample. In one embodiment, the depressed IP₃R mediated Ca²⁺ signals arise at the level of the IP3R channel, without a mutation in the IP₃R. In one embodiment, IP₃R Ca²⁺ signaling activity is measured by imaging Ca²⁺ flux though single ion channels within intact cells with single channel resolution, and wherein the method comprises using total internal reflection microscopy together with a slow Ca²⁺ buffer to restrict excitation of a cytosolic fluorescent Ca²⁺ indicator to within ˜100 nm of the plasma membrane; monitoring the local microdomain of elevated cytosolic [Ca²⁺] around the pore of Ca²⁺-permeable membrane channels; and dissecting the Ca²⁺ puffs arising from clusters of Ip3Rs by using localized single-channel Ca²⁺ fluorescence transients, wherein the single-channel Ca²⁺ fluorescence transients turn on and off rapidly, tracking channel openings and closings with a time resolution of a few ms. In one embodiment, a change in the Ca²⁺ signaling activity is determined through changes in the spatial distribution of IP₃R channels as imaged by super-resolution imaging. In one embodiment, IP₃R Ca²⁺ signaling activity is determined by an assay comprising monitoring cytosolic Ca²⁺ signals in skin fibroblasts from FXS and matched control subjects using a fluorimetric imaging plate reader; applying ATP to activate GPCR-linked purinergic P2Y receptors in Ca²⁺ free extracellular solution to exclude Ca²⁺ influx through plasmalemmel channels; and determining changes in IP₃R Ca²⁺ signaling activity.

In another embodiment, disclosed herein is a method for diagnosing susceptibility of autism spectrum disorder (ASD) in a subject, comprising the steps of providing a sample from the subject to be diagnosed; assaying the sample to determine IP₃R Ca²⁺ signaling activity levels; and comparing said signal activity level to a reference value based on the IP₃R Ca²⁺ signaling activity in a similar sample from a healthy control subject; wherein a lower activity level than the reference value in the sample is indicative of the presence of an autism spectrum disorder. In one embodiment, the method further comprises administering a ASD treatment to the subject. In one embodiment, the ASD treatment comprises administering a therapeutically effective dosage of a composition comprising one or more agonists of inositol triphosphate receptor (IP₃R) calcium (Ca²⁺) signaling. In one embodiment, the reduction of IP₃R Ca²⁺ signaling activity disrupts the normal mitochondrial bioenergetics, creating the energy deficient endophenotype of ASD.

Other features and advantages of the invention will become apparent from the following detailed description, taken in conjunction with the accompanying drawings, which illustrate, by way of example, various embodiments of the invention.

### DESCRIPTION OF THE DRAWINGS

FIG. 1 illustrates that Ca²⁺ responses to extracellular application of ATP in Ca²⁺-free solution are depressed in human skin fibroblasts from FXS patients as compared with matched controls. FIG. 1(a) displays representative FLIPR traces showing response to various concentrations of extracellular ATP (top panel) and to the Ca²⁺ ionophore ionomycin (lower panel) in control (Ctr) and FXS cells loaded with the Ca²⁺ indicator Fluo-8. Traces show fluorescence in arbitrary units, and each recording was obtained from a separate well. FIG. 1(b) demonstrates peak Ca²⁺ responses to 1 μM ionomycin in five control and five FXS cell lines. Bars show mean and SEM of triplicate measurements. FIG. 1(c) illustrates that cells from five FXS cell lines (grey bars) and matched controls (black bars) were stimulated with 100 μM ATP in Ca²⁺-free solution to stimulate Ca²⁺ release from intracellular Ca²⁺ stores. Recordings were performed in triplicate, averaged, and normalized with respect to corresponding ionomycin responses in Ca²⁺-free solution. n=3 in each group. FIG. 1(d) illustrates normalized Ca²⁺ responses to various concentrations of ATP derived by combining results from 5 FXS and 5 matched controls. All data in this and following figures are presented as mean±SEM; *=p-value <0.05; **=p<0.01 calculated from a two-sample Student's t-test.

FIG. 2 illustrates that Ca²⁺ responses were strongly depressed in TS1 and TS2 fibroblasts, but IP₃ receptor expression was not correlated with Ca²⁺ signal depression in TS or FXS cells. FIG. 2(a) displays representative FLIPR traces showing response to various concentrations of extracellular ATP (top panel) and to the Ca²⁺ ionophore ionomycin (lower panel) in control (Ctr) and TS cells loaded with the Ca²⁺ indicator Fluo-8. FIG. 2(b) demonstrates that three cell lines from TS patients (grey bars) and matched controls (black bars) were stimulated with 100 μM ATP in Ca²⁺-free solution to stimulate Ca²⁺ release from intracellular Ca²⁺ stores. Recordings were performed in triplicate, averaged, and normalized with respect to corresponding ionomycin responses in Ca²⁺-free solution. FIG. 2(c) shows normalized Ca²⁺ responses to various concentrations of ATP derived by combining results from three TS and three matched controls. n=3 replicates in each group. All data in this and following figures are presented as mean±SEM; *=p-value <0.05; **=p<0.01 calculated from a two-sample Student's t-test. FIG. 2(d) displays a scatter plot showing IP₃R expression levels in TS and FXS cell lines determined by western blotting versus the mean ATP-evoked Ca²⁺ signals in these cells relative to matched control cells. Different symbols represent different cell lines (TS2, downward arrow; TS1-B, circle; FXS-2, upward arrow; and FXS-4, square), and different colors represent IP₃R expression levels as determined using antibodies for type 1 (black), type 2 (red), type 3 (blue) IP₃Rs, and a non type-specific antibody (green). All data are normalized relative to matched control cells. Solid lines are regression fits to data for IP₃R1 (black), IP₃R2 (red), IP₃R3 (blue), and total IP₃Rs (green). The grey dashed line represents a one-to-one relationship between normalized Ca²⁺ signal and normalized IP₃R expression.

FIG. 3 demonstrates that Ca²⁺ release evoked by photoreleased IP₃ was depressed in FXS and TS cells. FIG. 3(a) displays representative frames taken from image sequences of control (top) and FXS fibroblasts (bottom) loaded with Fluo-8 and stimulated by photorelease of i-IP₃. Increasing cytosolic [Ca²⁺] (increasing fluorescence ratio % F/F₀) was depicted on a pseudocolor scale, as indicated by the color bar. Time-stamps indicated time from beginning of the record; the photolysis flash was delivered at 3 s. The monochrome panels on the left show resting fluorescence before stimulation to indicate cell outlines. FIG. 3(b) shows superimposed traces of representative global single-cell Ca²⁺ responses to uncaging of i-IP₃ in FXS (red) and control fibroblasts (black). Traces represented average fluorescence ratio signals (% F/Fₒ) throughout regions of interest encompassing the whole cell. Arrow indicated time of the UV flash. Data were from the cell pair labeled as FXS-2/Ctr-2 in FIG. 1(c). FIG. 3(c) illustrates that mean peak amplitude of Ca²⁺ responses was significantly depressed in FXS cells relative to matched controls. FIG. 3(d) shows that mean latency from time of photolysis flash to peak IP₃-evoked Ca²⁺ response was prolonged in FXS fibroblasts. FIG. 3(e) shows that mean rate of rise of Ca²⁺ fluorescence signal (peak amplitude/time to peak) was reduced in FXS cells as compared with control cells. Data in FIGS. 3(c)-3(e) were from 13 control cells and 14 FXS cells. FIGS. 3(f)-3(i) Corresponding traces FIG. 3(f), and mean values of amplitude FIG. 3(g), latency FIG. 3(h) and rate of rise FIG. 3(i) derived from cells labeled as Ctr-3 and TS1-B in FIG. 2c. Data are from 11 TS cells and 12 matched controls.

FIG. 4 illustrates Local IP₃-evoked Ca²⁺ events. FIG. 4(a) demonstrates resting Cal520 fluorescence of a control fibroblast (outlined) imaged by TIRF microscopy. Circles mark all sites where Ca²⁺ release events were identified within a 40 sec imaging record following photorelease of i-IP₃ in a 128×512 pixel (20.48×81.92 μm) imaging field. Larger circles mark sites from which traces in FIG. 4(b) were obtained. FIG. 4(b) show representative traces from sites numbered in FIG. 4(a). Dots underneath the traces marked events arising at that particular site; unmarked signals represented fluorescence bleed-through from events localized to adjacent but discrete sites. Arrow indicated the timing of the UV flash. FIG. 4(c) are examples of individual events shown on an expanded timescale to better illustrate their kinetics. FIG. 4(d) illustrates a surface intensity plot of three individual puffs near their peak times. FIG. 4(e) illustrates a single Ca²⁺ event shown on an expanded scale to illustrate measurements of peak amplitude and event duration at half-maximal amplitude.

FIG. 5 illustrates that IP₃-mediated Ca²⁺ signaling in FXS and TS fibroblasts was impaired at the level of local events. Data were from 17 FXS-3 cells, 17 TS1-B cells, and 16 control cells (Ctr-3) matched to both experimental groups. Open black squares in FIG. 5(a)-5(d) represented mean measurements from individual cells; histograms and error bars were overall means±1 SEM across all cells in each group. FIG. 5(a) illustrates total numbers of Ca²⁺ release sites detected within cells during 40 s imaging records following uniform photorelease of i-IP₃. FIG. 5(b) illustrates mean event frequency per site, calculated from the number of events observed per site throughout the recording period. FIG. 5(c) illustrates mean latencies following the photolysis flash to the first event at each site within a cell. FIG. 5(d) illustrates mean amplitudes of all events within each cell. FIG. 5(e) illustrates distributions of event durations (at half maximal amplitude) derived from all events identified in FXS (open diamonds), TS (stars) and control cells (black squares). The data were fit by single-exponential distributions with time constants tₒ of 15 ms (both FXS and TS) and 32 ms (control). Outcomes were compared using two-sample Mann-Whitney test. *=p-value <0.05; **=p<0.01, n/s—non-significant.

FIG. 6 illustrates Ca²⁺ responses to extracellular application of ATP in Ca²⁺-free solution are depressed in human skin fibroblasts from patients with syndromic and sporadic forms of ASD as compared with unaffected controls. Cells from 10 sporadic ASD patients, 7 FXS, 3 TS, 3 Rett and 3 Prader-Willi patients (grey bars) and 16 cell lines from unaffected controls (black bars) were stimulated with 100 μM ATP in Ca²⁺-free solution to stimulate Ca²⁺ release from intracellular Ca²⁺ stores. Recordings were performed in triplicate for each cell line, averaged, and normalized with respect to corresponding ionomycin responses in Ca²⁺-free solution. N for each group is indicated below each column.

FIG. 7 depicts, in accordance with various embodiments herein, additional data. (1) Ca²⁺ responses to extracellular application of ATP in Ca²⁺-free solution (1a) Representative FLIPR traces showing response to various concentrations of extracellular ATP (top) and Ca²⁺ ionophore ionomycin (bottom) in control (ctr) and FXS cells loaded with the Ca²⁺ indicator Fluo-8AM. (1b) Mean ATP-evoked Ca²⁺ signals in FXS (grey) and matched control (black) cell lines after normalizing as % of ionomycin response. (1c) Corresponding data from TSC1 and TSC2 cell lines. *p<0.05; **p<0.01. (1d) Scatter plot showing IP₃R expression levels in TS and FXS dell lines as % of matched controls vs. the mean ATP-evoked Ca²⁺ signals in these cells relative to matched controls. Different symbols represent different cell lines. (2) Methods—high throughput Ca²⁺ signaling. Skin fibroblasts were seeded in 96-well plates and loaded with 2 uM of Fluo—8AM. The assay was performed with a FLIPR instrument. 100 ul of 2×ATP in Ca²⁺-free HBSS was added to each well, along with addition of 100 ul of ionomycin to 1 uM final concentration. Fluorescence changes were normalized to ionomycin responses. Single cell Ca²⁺ imaging. Cells seeded in glass-bottomed dishes were loaded with 4 uM Fluo-8 AM and 1 uM i-IP₃ (ci-IP₃) of 45 minutes. [Ca²⁺] changes were imaged using a Nikon Eclipse microscope system with a 4× oil objective at 30 frames sec-1. A single flash of UV light from an arc lanp was used to uncage i-IP₃. For experiments studying local Ca²⁺ signals, cells were loaded with Ca²⁺ indicator, c-iIP₃, and additionally incubated with 10 um EGTA-AM for an hour [Ca²⁺] signals were imaged using Apo TIRP 100× (NA=1.49) oil objective. (3) IP₃ signaling is affected at the level of local events. (3a) traces of individual events. (3b) A single Ca²⁺ event showing peak amplitude and event duration at half-maximal amplitude. (3c) Total numbers of Ca²⁺ release sites following photorelease of i-IP3. (3d) Mean amplitudes of all events following the photolysis at each site. (3e) Distributions of event durations at half maximal amplitude derived from all events in FXS (open diamonds), TS (stars), and control cells (black squares). Time constants tm is 15 ms (both FXS and TS) and 32 ms (control). (4) Ca²⁺ signaling is decreased in syndromic and sporadic forms of autism spectrum disorder. Ca²⁺ responses to extracellular application of ATP in Ca2+-free solution are depressed in human skin fibroblasts from patients with syndromnic and sporadic forms of ASD (grey bars) as compared to unaffected controls (black bar).

FIG. 8 depicts, in accordance with various embodiments herein, hierarchical organization of Ca²⁺ signals; from fundamental single-channel events (‘blips’; A), to elementary events (‘puffs’; B) and global waves (C). Cartoons on the left illustrate the proposed spatial organization of IP₃R channels in the ER membrane that gives rise to these events, and traces at right are experimental fluorescence traces of blips, puff and wave.

FIG. 9 depicts, in accordance with various embodiments herein, optical single channel recording. (A) TIRF imaging of the local Ca²⁺ microdomain around an open IP₃R located in close proximity to the plasma membrane. (B) Comparison of puffs recorded by conventional wide-field fluorescence (grey) and by TIRF imaging with EGTA loaded (black). (C) Example of sites that show exclusively single-channel activity. (D) Fluorescence trace showing multiple puffs evoked at a single site following photorelease of IP₃. (E) Inset shows an individual puff recorded using the optical patch clamp on an expanded time scale illustrating step-wise changes in fluorescence arising from closings and openings of individual IP₃R channels. Histogram shows the distribution of step levels as multiples of the single-IP₃R channel (blip) fluorescence.

FIG. 10 depicts, in accordance with various embodiments herein, super-resolution STORM imaging of tubulin. (A). Conventional epifluorescence imaging of tubulin in a fixed BS-C-1 cell. (B). Single frame showing fluorescence of individual Alexa 647 molecules conjugated to an anti-tubulin antibody. (C), Super-resolution image of the cell in (A), constructed by locating the molecular positions of 50,000 frames like that in (B).

FIG. 11 depicts, in accordance with various embodiments herein, IP₃-mediated Ca²⁺ signaling in FXS and TS fibroblasts is impaired at the level of local events. Data are from 17 FXS-3 cells, 17 TS1-B cells, and 16 control cells (Ctr-3) matched to both experimental groups. (a) Representative traces of individual events to illustrate their kinetics. (b) A single Ca²⁺ event shown on an expanded scale to illustrate measurements of peak amplitude and event duration (τₒ) at half-maximal amplitude. (c) Distributions of event durations (at half maximal amplitude) derived from all events identified in FXS (open diamonds), TS (stars) and control cells (black squares). The data are fit by single-exponential distributions with time constants to of 15 ms (both FXS and TS) and 32 ms (control). *=p-value <0.05; **=p<0.01, n/s—non-significant. (d) Total numbers of Ca²⁺ release sites detected within cells during 40 s imaging records following uniform photorelease of i-IP₃. (e) Mean amplitudes of all events following the photolysis at each site within a cell.

FIG. 12 depicts, in accordance with various embodiments herein, reduced constitutive Ca²⁺ signals in FXS and elevated autophagy markers in ASD. (A) Locations of spontaneous Ca²⁺ signals in WT fibroblasts. (B) Ca²⁺ events from selected sites in A. (C) Numbers of sites in WT and FXS cells. (D) GFP-LC3 expression in WT cells showing ring-shaped structure characteristic of autophagosomes (E). Background-subtracted fluorescence of GFP-LC3 for WT, FXS, TSC2 fibroblasts. N=10 for all.

FIG. 13 depicts, in accordance with various embodiments herein, depression of Ca²⁺ responses to extracellular application of ATP in Ca²⁺-free solution in fibroblasts from patients with sporadic ASD. (A) Cells from 8 sporadic ASD patients (red) and age/gender-matched unaffected controls (black) were stimulated with 100 μM ATP. Recordings were performed in triplicate, averaged, and normalized to corresponding ionomycin responses. (B) Mean responses calculated by averaging data in A.

FIG. 14 depicts, in accordance with various embodiments herein, super-resolution STORM imaging of native IP₃R in COS-7 cells. (A) Plot depicts drift-corrected fluorophore localizations derived from a cell immunostained with a primary antibody raised against IP₃R and a secondary antibody custom labeled with Alexa Fluor 647. Scale bar=2 um. (B) Magnified and cropped IP₃R cluster footprints.

FIG. 15 depicts, in accordance with various embodiments herein, cAMP partially restores Ca²⁺ signaling in FXS cells (A), inhibiting control cells (B). Columns show global response to photorelease of IP₃ before (Ctr) and after 20-minute treatment with 25 μM 8-bromo-cAMP. Bars represent SEM.*p-value>0.05, **p-value<0.05 C. Inverse U-shape dependency of Ca²⁺ signaling on cAMP concentration.

FIG. 16 depicts, in accordance with various embodiments herein, basal mitochondrial respiration is depressed in ASD. Fibroblasts from multiple ASD patients were analyzed using the Seahorse XF to probe mitochondrial bioenergetics via oxygen consumption rate (OCR). PT1: sporadic ASD subject with very low ADOS score. PT2: sibling who scored higher, but within the ASD spectrum range. CTL: two non-affected control subjects.

FIG. 17 depicts, in accordance with various embodiments herein, derivation of neurons from human skin fibroblasts. (A) Differentiation of human iPSC to GABA interneurons involves 4 stages, including embryonic body (EB) formation, induction of neuroepithelial cells (NE), patterning of MGE progenitors and differentiating to GABA neurons. (B) Tuj1 staining of neuron-specific class III b-tubulin in differentiated human neuronal progenitors (red) and DAPI (blue) (C) Whole cell voltage recordings from iPSC derived neurons after 6 and 15 weeks in culture (top). Voltage-clamp records of Na⁺ and K⁺ currents (bottom). (D) Mean amplitudes (left) and time to peak (right) of Ca²⁺ responses in neuronal progenitor cells derived from control and FXS fibroblasts following photo-liberation of i-IP₃.

FIG. 18 depicts, in accordance with various embodiments herein, representative Ca²⁺ responses to extracellular application of ATP and ionomycin in absence of extracellular Ca²⁺ in fibroblasts from control and ASD patients. A. Representative FLIPR traces showing change in fluorescence (ΔF) in response to extracellular application of 100 μM ATP in a control (black traces) and ASD (red) cells loaded with the Ca²⁺ indicator Fluo-8. Traces show fluorescence in arbitrary units. B. Peak amplitude (ΔF) Ca²⁺ response to 100 μM ATP normalized to the basal fluorescence (F0) before stimulation in a control cell line (black) and an ASD line (red). C. Representative FLIPR traces showing change in fluorescence (ΔF) in response to extracellular application of 1 μM of the Ca²⁺ ionophore ionomycin in a control (black traces) and ASD (red) cells loaded with the Ca²⁺ indicator Fluo-8. Traces show fluorescence in arbitrary units. D. Peak Ca²⁺ response (ΔF) to 1 μM ionomycin normalized to the basal fluorescence (F0) before stimulation in a control cell line (black) and an ASD line (red). E. Peak amplitude (ΔF) Ca²⁺ response to 100 μM ATP normalized to the basal fluorescence (F0) before stimulation in a control cell line (black; N=12 patients), FXS cell line (dark grey; N=6 patients), Rett syndrome (light grey; N=2 patients), and TS (white bar; N=3 patients). Bar graphs show mean and SEM of triplicate measurements. F. Peak amplitude (ΔF) Ca²⁺ response to 1 μM ionomycin normalized to the basal fluorescence (F0) before stimulation. The same as E.

FIG. 19 depicts, in accordance with various embodiments herein, Ca²⁺ response in unaffected subjects and patients with various forms of ASD. A. Average Ca²⁺ response from unaffected controls (Ctr; N=12 patients), and sporadic ASD patients (ASD; N=29). Error bars represent SEM. **p-value <0.01. B. Average Ca²⁺ response in skin fibroblasts from unaffected controls (Ctr; N=12 patients), fragile X syndrome (FXS; N=6 patients), tuberous sclerosis syndrome (TS; N=3 patients), Rett syndrome (Rett; N=2 patients) and sporadic ASD (ASD; N=29) patients. Peak Ca²⁺ response (ΔF/F0) divided by the peak ionomycin response (ΔF/F0) was normalized to the mean value of the same reference cell line run on each plate. Horizontal streaks and error bars represent average of all cell lines in each category and SEM respectively. Individual data points represent individual cell line responses.

FIG. 20 depicts, in accordance with various embodiments herein, ROC results for Ca²⁺ signaling in ASD patients. A. ROC results for sporadic ASD patients and unaffected controls. The ROC graph represents sensitivity (i.e., true positive rate) plotted against 1—specificity (i.e., false positive rate). AUC is area under the ROC curve. B. ROC results for Ca²⁺ signaling in sporadic and syndromic ASD cohorts from (A). The ROC graph represents sensitivity (i.e., true positive rate) plotted against 1—specificity (i.e., false positive rate). AUC is area under the ROC curve.

FIG. 21 depicts, in accordance with various embodiments herein, Ca²⁺ release evoked by photoreleased IP3 is depressed in FXS and TS cells. Mean peak amplitude of Ca²⁺ responses is significantly depressed in FXS and TSC2 cells relative to matched controls.

### DETAILED DESCRIPTION OF THE DISCLOSURE

All references, publications, and patents cited herein are incorporated by reference in their entirety as though they are fully set forth. Unless defined otherwise, technical and scientific terms used herein have the same meaning as commonly understood by one of ordinary skill in the art to which this invention belongs. Hornyak, et al., Introduction to Nanoscience and Nanotechnology, CRC Press (2008); Singleton et al., Dictionary of Microbiology and Molecular Biology 3rd ed., J. Wiley & Sons (New York, N.Y. 2001); March, Advanced Organic Chemistry Reactions, Mechanisms and Structure 7th ed., J. Wiley & Sons (New York, N.Y. 2013); and Sambrook and Russel, Molecular Cloning: A Laboratory Manual 4th ed., Cold Spring Harbor Laboratory Press (Cold Spring Harbor, N.Y. 2012), provide one skilled in the art with a general guide to many of the terms used in the present application. One skilled in the art will recognize many methods and materials similar or equivalent to those described herein, which could be used in the practice of the present invention. Indeed, the present invention is in no way limited to the methods and materials described.

As described herein, the inventors developed an improved Ca²⁺ signaling assays to investigate the prevalence of signaling abnormalities across monogenic and sporadic forms of ASD. They have also determined the molecular mechanisms underlying the defect. Further, they have elucidated how IP₃R-mediated Ca²⁺ signaling deficits impact mitochondrial bioenergetics. Also, they extended their studies to Ca²⁺ signaling in neurons derived from induced pluripotent stem cells (iPSCs) cells generated from fibroblasts from monogenic and sporadic ASD subjects.

In one embodiment, the present invention provides a method of diagnosing a risk for a patient developing autism spectrum disorder (ASD) comprising identifying a reduced inositol triphosphate receptor (IP₃R) calcium (Ca²⁺) signaling activity level in cells from the patient compared to matched cells from a control individual, and diagnosing a risk of the patient developing ASD when the reduced IP₃R activity level is identified, wherein the control individual is an individual without ASD. In another embodiment, the cells are skin fibroblast cells and/or amniocyte obtained prenatally by amniocentesis. In another embodiment, the identifying the reduced IP₃R Ca²⁺ signaling activity level further comprises: (a) obtaining equivalent amounts of separately cultured skin fibroblast cells from the patient and from the control individual, wherein the cultured skin fibroblast cells from each of the patient and the control individual have been loaded with a Ca²⁺ fluorescent probe, and contacted with an agonist of IP₃R Ca²⁺ signaling; (b) measuring, in each of the cultured skin fibroblast cells from the patient and the individual obtained in (a), an amount of fluorescence emitted by the Ca²⁺fluorescent probe; and (c) comparing the amounts of emitted fluorescence measured in (b).

In another embodiment, the present invention is a method of identifying a therapeutic anti-ASD agent comprising of: (a) loading each of two populations of isolated cells with a Ca²⁺ fluorescent probe; (b) contacting each of the first population of isolated cells and the second population of isolated cells with an agonist of IP₃R Ca²⁺ signaling; (c) exposing the first population of isolated cells to a test agent; (d) measuring fluorescence emitted by the fluorescent Ca²⁺ indicator in the first population of isolated cells to determine a test IP₃R Ca²⁺ signaling activity; (e) measuring an amount of fluorescence emitted by the Ca²⁺ fluorescent probe in the second population of isolated cells to determine a test IP₃R Ca²⁺ signaling activity; and (f) detecting a difference between the test and the control IP₃R Ca²⁺ signaling activities, wherein an increased IP₃R Ca²⁺ signaling activity in the first population of isolated cells as compared to the second population of isolated cells detected in (f) identifies the test agent as a potentially therapeutic anti-ASD agent.

In one embodiment, the inventors have found, in studies on skin cells (fibroblasts) derived from affected patients, that inositol trisphosphate (IP₃)-induced Ca2+ response is significantly diminished in fragile X syndrome (FXS) and tuberous sclerosis (TS)—two genetic diseases with high co-morbidity with ASD. Moreover, cells from patients with non-syndromic forms of ASD also revealed a greatly diminished Ca²⁺ response, making IP₃-mediated Ca²⁺ signaling a widely shared signaling abnormality in ASD. Ca²⁺ screening in skin fibroblasts offers a technique in conjunction with behavioral testing for early detection of ASD, and for high-throughput screening of novel therapeutic agents.

In one embodiment, the disclosure herein allows diagnosis of autism based on a biological test that can be done in a laboratory. It is qualitatively different from behavioral tests that use currently used. It can identify children with predisposition to autism earlier than alternative methods currently used. Additionally, in accordance with various embodiments herein, assays described herein are a cheaper and faster form of screening for possible therapeutic agents for treatment of autism.

In another embodiment, the present invention provides a method of treating a disease by identifying abnormal inositol triphosphate receptor (IP₃R) calcium (Ca²⁺) signaling in an individual, and treating the individual. In another embodiment, the abnormal inositol triphosphate receptor (IP₃R) calcium (Ca²⁺) signaling is a reduced level in activity of inositol triphosphate receptor (IP₃R) calcium (Ca²⁺) signaling. In another embodiment, treating the disease comprises administering a therapeutically effective dosage of a composition comprising one or more agonists of inositol triphosphate receptor (IP₃R) calcium (Ca²⁺) signaling. In another embodiment, the disease is ASD.

As described herein, the inventors have disclosed in detail disrupted IP₃-mediated Ca²⁺ signaling as a ubiquitous phenotype across multiple diverse forms of ASD, both monogenic and sporadic. Moreover, the inventors have investigated how Ca²⁺ signaling abnormalities in skin cells from diverse deeply phenotyped subjects with ASD can serve as a potential biomarker to be used in the diagnosis of ASD. Further, the inventors have elucidated the mechanistic defects in IP3R channel function and their consequent effects on mitochondrial bioenergetics by utilizing advanced biophysical and imaging technologies. Also, the inventors have disclosed their development and studies of iPSC-derived neurons from the same ASD subjects.

In one embodiment, disclosed herein is a kit for determining susceptibility to ASD, comprising an assay for determining an increase or decrease of IP₃R Ca²⁺ signaling activity level in cells, wherein a decrease in IP₃R Ca²⁺ activity is indicative of ASD susceptibility. In one embodiment, the IP₃R Ca2+ signaling activity is determined by using one or more biomarkers that is a clinically tractable discriminant of ASD. In one embodiment, the ASD is monogenic ASD and/or sporadic ASD. In one embodiment, the monogenic form of ASD comprises FXS, TSC1, and/or TSC2. In one embodiment, the cells comprise fibroblast cells or neuronal cells. In one embodiment, the decrease of IP₃R Ca²⁺ signaling activity arises at the IP3R channel, without a mutation in the IP₃R. In one embodiment, IP₃R Ca²⁺ signaling activity is measured by imaging Ca²⁺ flux though single ion channels within intact cells with single channel resolution. In one embodiment, IP₃R Ca²⁺ signaling activity is measured by the following: using total internal reflection microscopy together with a slow Ca²⁺ buffer to restrict excitation of a cytosolic fluorescent Ca²⁺ indicator to within ˜100 nm of the plasma membrane; monitoring the local microdomain of elevated cytosolic [Ca²⁺] around the pore of Ca²⁺-permeable membrane channels; and dissecting the Ca²⁺ puffs arising from clusters of IP₃Rs by using localized single-channel Ca²⁺ fluorescence transients, wherein the single-channel Ca²⁺ fluorescence transients turn on and off rapidly, tracking channel openings and closings with a time resolution of a few ms. In one embodiment, a change in the Ca²⁺ signaling activity is determined through changes in the spatial distribution of IP3R channels as imaged by super-resolution imaging. In one embodiment, IP₃R Ca²⁺ signaling activity is determined by an assay comprising monitoring cytosolic Ca²⁺ signals in skin fibroblasts from FXS and matched control subjects using a fluorimetric imaging plate reader; applying ATP to activate GPCR-linked purinergic P2Y receptors in Ca²⁺ free extracellular solution to exclude Ca²⁺ influx through plasmalemmel channels; and determining changes in IP₃R Ca²⁺ signaling activity. In one embodiment, identifying the reduced IP₃R Ca²⁺ signaling activity level further comprises obtaining equivalent amounts of separately cultured skin fibroblast cells from the patient and from the control individual, wherein the cultured skin fibroblast cells from each of the patient and the control individual have been loaded with a Ca²⁺ fluorescent probe, and contacted with an agonist of IP₃R Ca²⁺ signaling; measuring, in each of the cultured fibroblast cells from the patient and the individual obtained in (a), an amount of fluorescence emitted by the Ca²⁺ fluorescent probe; and comparing the amounts of emitted fluorescence measured in (b).

In another embodiment, disclosed herein is a method of screening a therapeutic agent for ASD comprising providing a cell sample of a subject diagnosed with ASD; detecting the IP₃R Ca²⁺ signaling activity in the cell sample in the presence, as well as the absence of a candidate drug; and determining that the candidate drug is a therapeutic agent for treatment of ASD if the IP₃R Ca²⁺ signaling activity is higher in the presence of the candidate drug than in its absence. In one embodiment, the ASD is monogenic ASD or sporadic ASD. In one embodiment, the monogenic form of ASD comprises FXS, TSC1, and/or TSC2. In one embodiment, the cell sample comprises a skin fibroblast cell sample, an amniocyte cell sample obtained prenatally by amniocentesis, or a neuronal cell sample. In one embodiment, the depressed IP₃R mediated Ca²⁺ signals arise at the level of the IP₃R channel, without a mutation in the IP₃R. In one embodiment, IP₃R Ca²⁺ signaling activity is measured by imaging Ca²⁺ flux though single ion channels within intact cells with single channel resolution, and wherein the method comprises using total internal reflection microscopy together with a slow Ca²⁺ buffer to restrict excitation of a cytosolic fluorescent Ca²⁺ indicator to within ˜100 nm of the plasma membrane; monitoring the local microdomain of elevated cytosolic [Ca²⁺] around the pore of Ca²⁺-permeable membrane channels; and dissecting the Ca²⁺ puffs arising from clusters of IP₃Rs by using localized single-channel Ca²⁺ fluorescence transients, wherein the single-channel Ca²⁺ fluorescence transients turn on and off rapidly, tracking channel openings and closings with a time resolution of a few ms. In one embodiment, a change in the Ca²⁺ signaling activity is determined through changes in the spatial distribution of IP₃R channels as imaged by super-resolution imaging. In one embodiment, IP₃R Ca²⁺ signaling activity is determined by an assay comprising monitoring cytosolic Ca²⁺ signals in skin fibroblasts from FXS and matched control subjects using a fluorimetric imaging plate reader; applying ATP to activate GPCR-linked purinergic P2Y receptors in Ca²⁺ free extracellular solution to exclude Ca²⁺ influx through plasmalemmel channels; and determining changes in IP₃R Ca²⁺ signaling activity.

In another embodiment, disclosed herein is a method for prognosis, diagnosis, or treatment for an autism spectrum disorder (ASD), comprising the steps of providing a biological sample from the subject to be diagnosed; determining IP₃R Ca²⁺ signaling activity level in the said biological sample; and comparing said signal activity level to a reference value based on the IP₃R Ca²⁺ signaling activity in a similar sample from a healthy control subject; wherein a lower activity level than the reference value in the sample is indicative of the presence of an autism spectrum disorder. In one embodiment, the method further comprises administering a ASD treatment to the subject. In one embodiment, the ASD treatment comprises administering a therapeutically effective dosage of a composition comprising one or more agonists of inositol triphosphate receptor (IP₃R) calcium (Ca²⁺) signaling. In one embodiment, the reduction of IP₃R Ca²⁺ signaling activity disrupts the normal mitochondrial bioenergetics, creating the energy deficient endophenotype of ASD.

As described herein, the inventors found that IP₃-mediated Ca²⁺ release from the endoplasmic reticulum in response to activation of purinergic receptors is significantly depressed in patients with both rare syndromic and sporadic forms of ASD. This defect is not due to the different endoplasmic reticulum Ca²⁺ content, as judged from the response to ionomycin, a Ca²⁺ ionophore. The inventors have identified a highly prevalent functional signaling defect in a cohort of diverse patients with ASD that holds promise as a biomarker for diagnosis and novel drug discovery. These results illustrate that deficits in IP₃-mediated Ca²⁺ signaling is likely to be a convergent hub function shared across different forms of ASD whether caused by rare highly penetrant mutations or sporadic forms.

In one embodiment, disclosed herein is a method of diagnosing ASD in a subject comprising activating purinergic receptors in a cell sample of the subject; measuring the IP₃-mediated Ca²⁺ release from the endoplasmic reticulum of the cell sample; and diagnosing ASD in the subject if IP₃-mediated Ca²⁺ release is depressed compared to a healthy control subject without ASD. In one embodiment, the ASD is a syndromic or a sporadic form. In one embodiment the depressed level of Ca²⁺ release is not due to the different endoplasmic reticulum Ca2+ content, as judged from the response to ionomycin, a Ca²⁺ ionophore.

Some embodiments of the present invention is directed to a kit for determining susceptibility to Autism Spectrum Disorder (ASD. The kit is useful for practicing the inventive method of diagnostics of autism. The kit is an assemblage of materials or components, including at least one of the inventive compositions. Thus, in some embodiments the kit contains a composition including an assay for determining an increase or decrease of IP₃R Ca²⁺ signaling activity levels in a cell, as described above.

The exact nature of the components configured in the inventive kit depends on its intended purpose. For example, some embodiments are configured for the purpose of treating or diagnosing autism. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to determine susceptibility to ASD or treatment of ASD. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in the medical and therapeutic field. As used herein, the term “package” refers to a suitable solid matrix or material such as glass, plastic, paper, foil, and the like, capable of holding the individual kit components. Thus, for example, a package can be a glass vial used to contain suitable quantities of an inventive composition containing an assay for determining an increase or decrease of IP₃R Ca²⁺ signaling activity levels in a cell. The packaging material generally has an external label which indicates the contents and/or purpose of the kit and/or its components.

In various embodiments, the present invention utilizes biomarkers and the detection of biomarkers, such as for detecting and measuring the presence of and activity of IP₃R Ca²⁺ signaling activity. There are many techniques readily available in the field for detecting the presence or absence of polypeptides or other biomarkers, including protein microarrays. For example, some of the detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

Similarly, there are any number of techniques that may be employed to isolate and/or fractionate biomarkers. For example, a biomarker may be captured using biospecific capture reagents, such as antibodies, aptamers or antibodies that recognize the biomarker and modified forms of it. This method could also result in the capture of protein interactors that are bound to the proteins or that are otherwise recognized by antibodies and that, themselves, can be biomarkers. The biospecific capture reagents may also be bound to a solid phase. Then, the captured proteins can be detected by SELDI mass spectrometry or by eluting the proteins from the capture reagent and detecting the eluted proteins by traditional MALDI or by SELDI. One example of SELDI is called “affinity capture mass spectrometry,” or “Surface-Enhanced Affinity Capture” or “SEAC,” which involves the use of probes that have a material on the probe surface that captures analytes through a non-covalent affinity interaction (adsorption) between the material and the analyte. Some examples of mass spectrometers are time-of-flight, magnetic sector, quadrupole filter, ion trap, ion cyclotron resonance, electrostatic sector analyzer and hybrids of these.

Alternatively, for example, the presence of biomarkers such as polypeptides maybe detected using traditional immunoassay techniques. Immunoassay requires biospecific capture reagents, such as antibodies, to capture the analytes. The assay may also be designed to specifically distinguish protein and modified forms of protein, which can be done by employing a sandwich assay in which one antibody captures more than one form and second, distinctly labeled antibodies, specifically bind, and provide distinct detection of, the various forms. Antibodies can be produced by immunizing animals with the biomolecules. Traditional immunoassays may also include sandwich immunoassays including ELISA or fluorescence-based immunoassays, as well as other enzyme immunoassays.

Prior to detection, biomarkers may also be fractionated to isolate them from other components in a solution or of blood that may interfere with detection. Fractionation may include platelet isolation from other blood components, sub-cellular fractionation of platelet components and/or fractionation of the desired biomarkers from other biomolecules found in platelets using techniques such as chromatography, affinity purification, 1D and 2D mapping, and other methodologies for purification known to those of skill in the art. In one embodiment, a sample is analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

As understood by one of skill in the art, Autism Spectrum Disorder (ASD) is a complex heterogeneous disorder with a poorly defined etiology and diagnosis criteria that are strictly clinical because there are as yet no objective biomarkers of the disorder. ASD also includes the disorder generally known as Autism.

### EXAMPLES

### Example 1: Materials

The membrane permeant caged IP₃ analogue ci-IP₃/PM (D-2,3-O-Isopropylidene-6-O-(2-nitro-4,5-dimethoxy)benzyl-myo-Inositol 1,4,5-trisphosphate-Hexakis (propionoxymethyl) ester) was obtained from SiChem (Bremen, Germany), diluted in 20% pluronic F-127 solution in DMSO to a stock concentration of 200 μM and was frozen down into 2 μl aliquots until needed. EGTA-AM and pluronic F-127 were from Molecular Probes/Invitrogen (Carlsbad, Calif.). Fluo-8 AM and Cal520 were purchased from AAT Bioquest.

### Example 2: Fibroblast Cells

Primary, untransformed human skin fibroblasts were purchased from Coriell Cell Repository. ASD cell lines and matched controls with their corresponding Coriell numbers are as follows: FXS-1 (GM05848)/Ctr-1 (GM00498), FXS-2 (GM09497)/Ctr-2 (GM02912), FXS-3 (GM05185)/Ctr-3 (GM03440), FXS-4 (GM04026)/Ctr-4 (GM02185), FXS-5 (GM05131)/Ctr-5 (GM05659), TS1-A (GM06148)/Ctr-6 (GM01863), TS1-B (GM06149)/Ctr-3 (GM03440), TS2 (GM06121)/Ctr-2 (GM02912). All cell lines came from male Caucasian patients. Cells were cultured in Dulbecco's Modified Eagle's Media (ATCC 30-2002) supplemented with 10% (v/v) fetal bovine serum and 1× antibiotic mix (penicillin/streptomycin) at 37° C. in a humidified incubator gassed with 95% air and 5% CO₂, and used for up to 15 passages. Cells were harvested in Ca²⁺, Mg²⁺-free 0.25% trypsin-EGTA (Life Technologies) and sub-cultured for 2 days before use.

### Example 3: High-Throughput Ca²⁺ Imaging

Skin fibroblasts were seeded in clear-bottom black 96-well plates (Greiner Bio One T-3026-16) at 1.3×10⁴ cells per well and grown to confluency. On the day of the experiment, cells were loaded by incubation with 2 μM of the membrane-permeant Ca²⁺ indicator Fluo-8 AM⁴⁶ in standard buffer solution (130 mM NaCl, 2 mM CaCl₂, 5 mM KCl, 10 mM glucose, 0.45 mM KH₂PO₄, 0.4 mM Na₂HPO₄, 8 mM MgSO₄, 4.2 mM NaHCO₃, 20 mM HEPES and 10 μM probenecid) with 0.1% fetal bovine serum for 1 h at 37° C., then washed with a standard buffer solution. Ca²⁺-free solution (120 mM NaCl, 4 mM KCl, 2 mM MgCl₂, 10 mM glucose, 10 mM HEPES, 1 mM EGTA) was added to each well (100 μl), and cells were allowed to equilibrate for 5 minutes prior to assay with a Fluorometric Imaging Plate Reader (FLIPR; Molecular Devices, Sunnyvale, Calif.). A basal read of fluorescence in each well (470-495 nm excitation and 515-575 nm emission, expressed in arbitrary units; AU) was read for 2 seconds. Next, 100 μl of 2×ATP (1 μM, 10 μM, 100 μM final concentration) or 100 μl of 2× ionomycin (to 1 μM final concentration) in Ca²⁺-free HBSS was added to each well. Only a single recording was obtained from a given well. Ionomycin-induced fluorescence changes from wells without prior addition of ATP were used to normalize ATP-evoked responses. Recordings were performed in triplicate.

### Example 3: Whole-Cell Ca²⁺ Imaging

Cells seeded in glass-bottomed dishes were loaded for imaging using membrane-permeant esters of Fluo-8 and caged i-IP₃ (ci-IP₃). Briefly, cells were incubated at room temperature in HEPES-buffered saline (2.5 mM CaCl₂, 120 mM NaCl, 4 mM KCl, 2 mM MgCl₂, 10 mM glucose, 10 mM HEPES) containing 1 μM ci-IP₃/PM for 45 mins, after which 4 μM Fluo-8 AM was added to the loading solution for further 45 minutes before washing three times with the saline solution. [Ca²⁺]ᵢ changes were imaged using a Nikon Eclipse microscope system with a 40× (NA=1.30) oil objective. Fluo-8 fluorescence was excited by 488 nm laser light, and emitted fluorescence (lambda >510 nm) was imaged at 30 frames sec⁻¹ using an electron-multiplied CCD Camera iXon DU897 (Andor). A single flash of UV (ultraviolet) light (350-400 nm) from an arc lamp focused to uniformly illuminate a region slightly larger than the imaging field was used to uncage i-IP₃, a metabolically stable isopropylidene analogue of IP₃, which evoked activity persisting for a few minutes. Image data were acquired as stack.nd2 files using Nikon Elements for offline analysis. Fluorescence signals are expressed as a ratio (ΔF/F₀) of changes in fluorescence (ΔF) relative to the mean resting fluorescence at the same region before stimulation (F₀). Recordings were performed in triplicate, and the measurement outcomes were compared using Mann-Whitney test.

### Example 4: Imaging Local Ca²⁺ Events

For experiments studying local Ca²⁺ signals, cells were incubated at room temperature in HEPES buffer containing 1 uM ci-IP₃/PM and 4 □M Cal520 for one hour⁴⁸, washed and further incubated with 10 uM EGTA AM for an hour. Cells were then washed three times and remained in buffer for 30 min to allow for de-esterification of loaded reagents. [Ca²⁺]ᵢ signals were imaged using the Nikon Eclipse microscope system described above, but now utilizing an Apo TIRF 100× (NA=1.49) oil objective. The imaging region on the camera sensor was cropped to 128×512 pixels (20.48×81.92 μm) to enable rapid (129 frames per second) imaging. Cal520 fluorescence (lambda >510 nm) was excited by 488 nm laser light within an evanescent field extending a few hundred nanometers into the cells. Image acquisition and processing was as described above for whole-cell imaging, except that local events were identified and analyzed using a custom-written algorithm based on MatLab.

### Example 5: Western Blot Analysis

Cell lines were grown in triplicates and lysed in mammalian protein extraction reagent (Thermo Scientific) with complete mini protease inhibitor cocktail tablets (Roche) and phosphatase 2 inhibitor cocktail (Sigma-Aldrich). Lysates were subsequently centrifuged at 14,000 rpm for 15 minutes at +4° C. Protein levels in the cell lysate were measured using the Bradford method. 20 μg of protein was loaded per well with 5% β-mercaptoethanol on 3%-8% gradient Tris-Acetate gels with Tris-Acetate SDS running buffer (Invitrogen) and separated by electrophoresis at 130V. Proteins were transferred at 50 mA for 6 hours to 0.2 μm nitrocellulose membranes, which were blocked in 5% nonfat milk in tris-buffered saline supplemented with 0.1% tween-20 for 1 hr. Membranes were probed overnight at +4° C. with the following primary antibodies: rabbit polyclonal anti-IP₃R1 (Millipore, AB5882), rabbit polyclonal anti-IP₃R2 (LifeSpan Biosciences, LS-C24911), mouse monoclonal anti-IP₃R3 (BD Transduction Laboratories, 610312), rabbit polyclonal anti-IP₃R1/2/3 (Santa-Cruz Biotechnology, sc-28613), rabbit polyclonal anti-beta actin (Abcam, ab8227). Membranes were then incubated, as appropriate, with goat anti-rabbit (1:5,000, Sigma-Aldrich) or goat anti-mouse (1:5,000, Sigma-Aldrich) HRP-conjugated secondary antibodies for 1 hr. Bands were visualized by an ImageQuant LAS 4000 imager (GE Healthcare) using peroxidase substrate for enhanced chemiluminescence (ECL Prime; Amersham). Levels of protein expression were quantified via densitometry analysis using ImageJ, and are expressed normalized to actin levels.

### Example 6: Agonist-Induced Ca²⁺ Signaling is Depressed in FXS and TS Fibroblasts

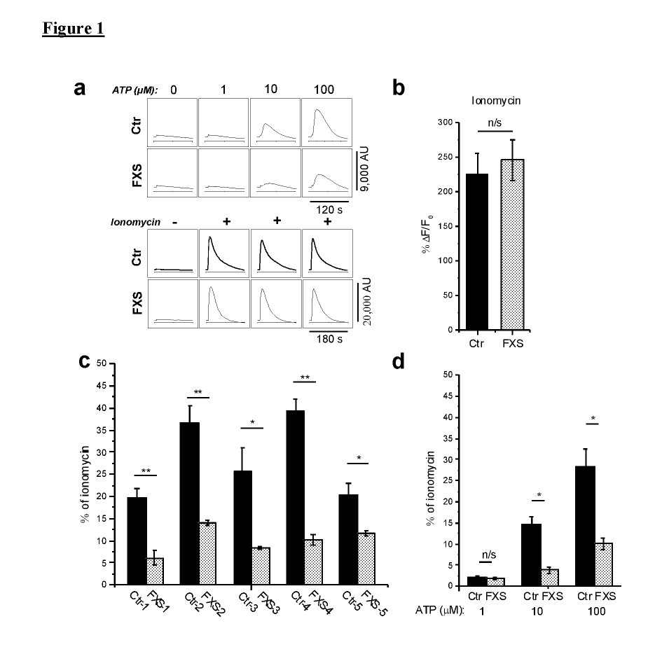

To screen for defects in IP₃-mediated signaling associated with ASD, a fluorometric imaging plate reader (FLIPR) was used to monitor cytosolic Ca²⁺ changes in fibroblasts loaded with the Ca²⁺-sensitive fluorescent indicator Fluo-8. Primary skin fibroblasts derived from five FXS males and five ethnicity- and age-matched unaffected male donors were grown to confluency on 96 well plates. Cells were stimulated by application of ATP to activate purinergic P2Y receptors and thereby evoke GPCR-mediated intracellular Ca²⁺ release through IP₃Rs. Recordings were made in Ca²⁺-free extracellular solution to exclude complication from Ca²⁺ influx through plasmalemmal channels. Different concentrations of ATP were applied to individual wells containing FXS and matched control cells. FIG. 1a (top panel) illustrated representative results, showing smaller ATP-evoked Ca²⁺ signals in FXS cells. To determine whether differences in ATP-evoked signals may result from differences in filling of ER Ca²⁺ stores, signals evoked in separate wells were recorded by application of 1 μM ionomycin in Ca²⁺-free medium to completely liberate all intracellular Ca²⁺ stores (FIG. 1a, lower panel). No significant difference was observed between mean ionomycin-evoked Ca²⁺ signals in FXS and control cells (FIG. 1b), suggesting that there was no systematic defect in ER Ca²⁺ store filling in FXS cells. To normalize for differences in store content among different cell lines and experimental days, ATP-evoked signals were expressed as a percentage of the ionomycin response obtained in parallel measurements in the same 96 well plate for each given cell line. Mean normalized Ca²⁺ signals evoked by 100 μM ATP were significantly depressed in all five FXS fibroblast lines in comparison with their matched controls (FIG. 1c). A similar depression was observed at lower concentrations of ATP, pooling data across all 5 FXS and control cell lines (FIG. 1d). These results were consistently reproducible across different experimental days and matched cell pairs (total of 12 paired trials).

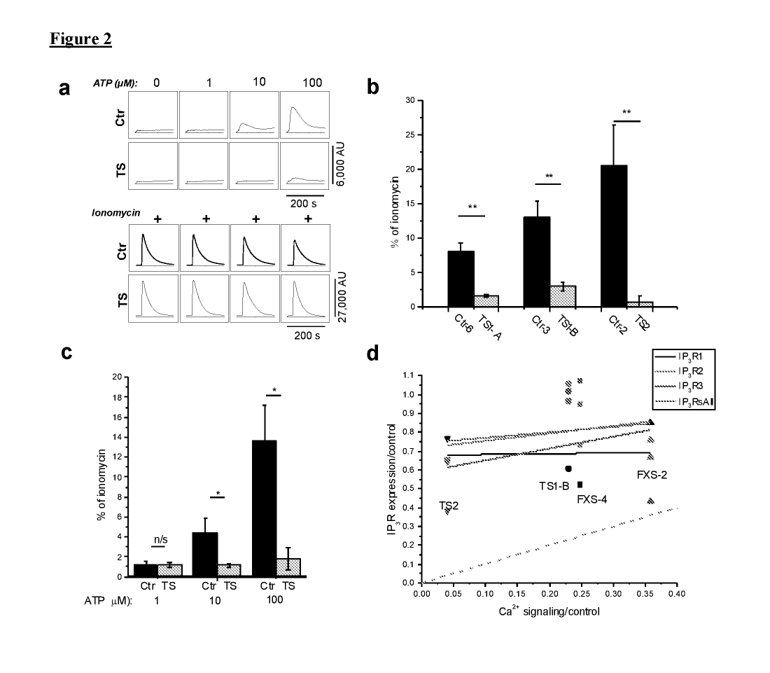

The findings were extended to another genetic disorder with high co-morbidity with ASD, tuberous sclerosis (TS), caused by mutations in either of two distinct and independent genes—hamartin (TSC1) or tuberin (TSC2). FIG. 2 shows data obtained by FLIPR screening in the same way as performed for FIG. 1. Three cell lines derived from TS patients demonstrated a consistent and highly significant deficit in ATP-evoked Ca²⁺ signals as compared with matched controls (FIGS. 2a,b,c), but without any appreciable difference in intracellular Ca²⁺ store content as assessed by ionomycin application (FIG. 2a, lower panel). These findings were consistently replicated on different experimental days (total of 6 paired trials).

To investigate whether the diminished Ca²⁺ signals in FXS and TS cells resulted from lower expression levels of IP₃R proteins, western blot analysis was performed on four cell lines selected as showing pronounced defects in Ca²⁺ signaling (FXS-2, FXS-4, TS1-B, and TS2), together with three matched control lines (Ctr-2, Ctr-3, Ctr-4), using antibodies specific to type 1, 2 and 3 IP₃Rs as well as a non type-specific antibody. The results showed an overall slight decrease in IP₃R expression across all isotypes in FXS and TS cells relative to their matched controls (FIG. 2d). However, in all cases the depression of IP₃R expression was much smaller than the corresponding depression of Ca²⁺ signaling as measured in the FLIPR experiments, and there was little or no correlation between IP₃R expression and Ca²⁺ signaling in the TS and FXS cells after normalizing relative to their matched controls (FIG. 2d).

### Example 7: IP₃-Induced Ca²⁺ Release is Reduced in FXS and TS Cells

To discriminate whether the observed deficits in ATP-induced Ca²⁺ signals in FXS and TS cell lines arose through defects in any of the intermediate steps from binding to purinergic GPCR receptors to generation of IP₃, or at the level of IP₃-mediated Ca²⁺ liberation itself, upstream GPCR signaling was circumvented by loading cells with a caged analogue of IP₃ (ci-IP₃). UV flash photolysis of ci-IP₃ to photorelease physiologically active i-IP₃ allowed to directly evoke Ca²⁺ liberation through IP₃Rs in a graded manner by regulating flash duration and intensity to control the amount of i-IP₃ that was photoreleased.

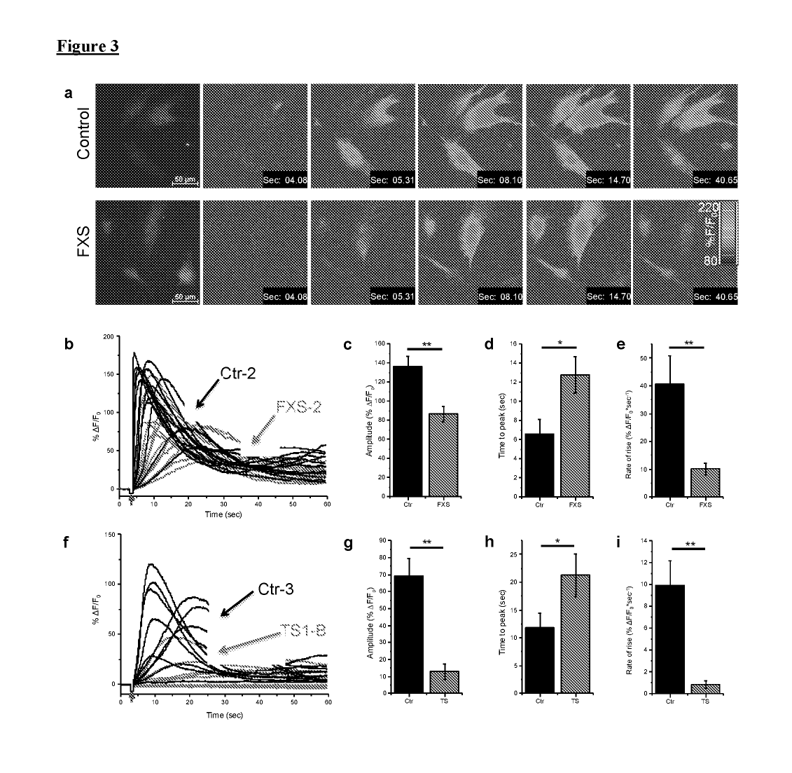

FIG. 3a illustrates images obtained by epifluorescence microscopy of FXS and control fibroblasts loaded with Fluo-8 and caged i-IP₃ by incubation with membrane-permeant esters of these compounds. FIG. 3b shows superimposed fluorescence ratio (ΔF/Fₒ) traces measured from several representative FXS-2 and matched control Ctr-2 cells in response to uniform photolysis flashes. Concordant with the observations of defects in ATP-induced global Ca²⁺ signals, global cytosolic Ca²⁺ responses evoked by equivalent photorelease of i-IP₃ in these FXS cells were smaller than in control cells (FIG. 3c); and displayed a longer time to peak (FIG. 3d) and slower rate of rise (FIG. 3e). Similar results were obtained from two other FXS-Ctr cell pairs (FXS-1/Ctr-1: 20.7±3.9/44.6±12.2% ΔF/F₀, FXS-3/Ctr-3: 20.1±4.8/156.8±17.3). Moreover, a consistent proportional depression of Ca²⁺ signals for different relative UV flash strengths corresponding to photorelease of different i-IP₃ concentrations was observed (25% flash strength, pooled FXS response 61% of control; 50% flash, 65% of control; 100% flash, 74% of control: n=13-17 cells for each flash duration).

TS cells also showed depressed and slowed Ca²⁺ responses to photoreleased i-IP₃. Measurements from the matched TS1-B and Ctr-3 cell lines (FIG. 3f) revealed a pronounced deficit in average Ca²⁺ signal amplitudes (FIG. 3g); and again the time to peak was lengthened (FIG. 3h) and the rate of rise slowed (FIG. 3i). These differences were apparent employing two different relative UV flash strengths (15% flash strength, TS response 18% of control; 25% flash, 20% of control: n=13-15 cells for each flash duration).

### Example 8: IP₃-Signaling is Affected at the Level of Local Events

IP₃-mediated cellular Ca²⁺ signaling is organized as a hierarchy, wherein global, cell-wide signals, such as those discussed above, arise by recruitment of local, ‘elementary’ events involving individual IP₃R channels or clusters of small numbers of IP₃Rs.

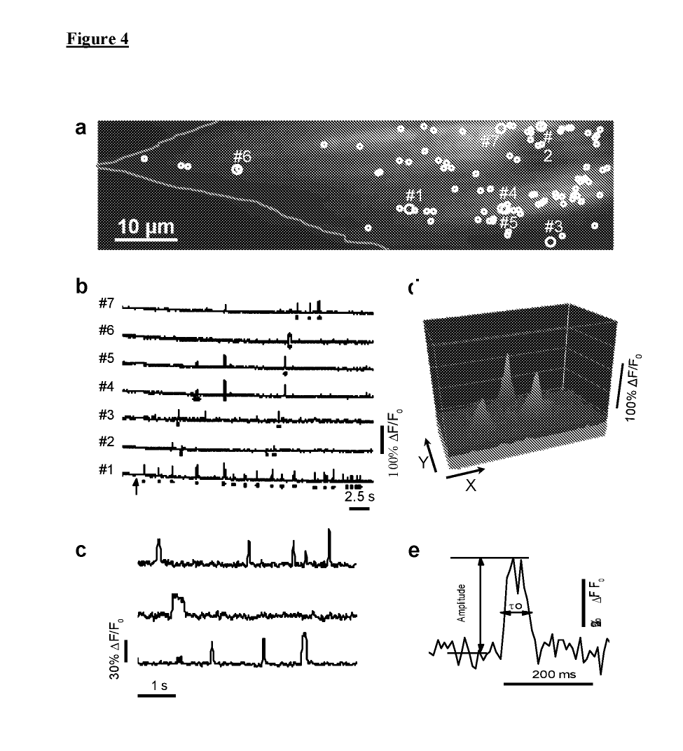

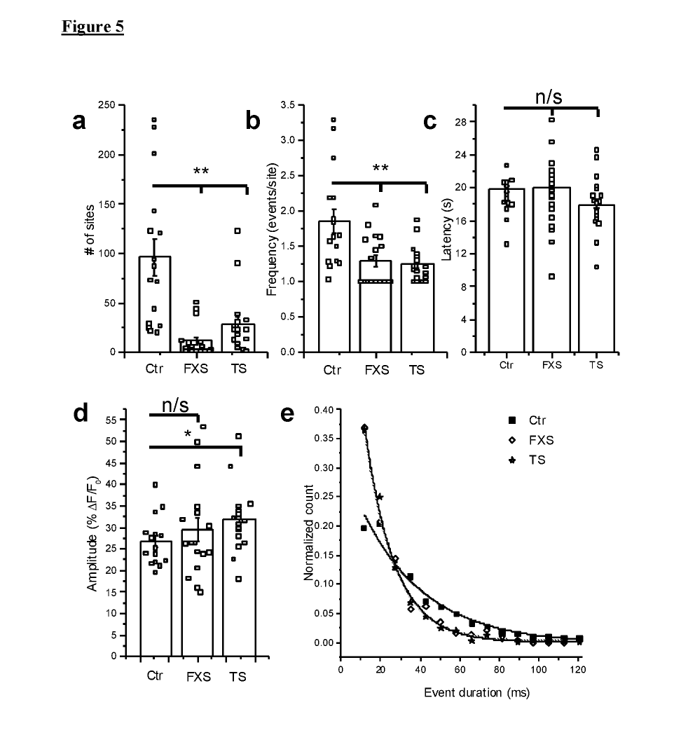

These elementary events were imaged to elucidate how deficits in the global Ca²⁺ signals in FXS and TS cells arises at the level of local IP₃R clusters. One FXS (FXS-3) fibroblast line, one TS1 (TS1-B) line, and a common control (Ctr-3) cell line matched to both was selected. Ca²⁺ release from individual sites was resolved utilizing total internal reflection fluorescence (TIRF) microscopy of Cal520 (a Ca²⁺ indicator that provides brighter signals than Fluo-4), in conjunction with cytosolic loading of the slow Ca²⁺ buffer EGTA to inhibit Ca²⁺ wave propagation. This technique captured in real time the duration and magnitude of the underlying Ca²⁺ flux, providing a close approximation of the channel gating kinetics as would be recorded by electrophysiological patch-clamp recordings. Ca²⁺ release evoked by spatially uniform photolysis of ci-IP₃ across the imaging field was apparent as localized fluorescent transients of varying amplitudes, arising at numerous discrete sites widely distributed across the cell body (FIG. 4a). Representative fluorescence traces illustrating responses at several sites (marked by large circles in FIG. 4a) are shown in FIG. 4b; and FIGS. 4c,d respectively illustrate the time course and spatial distribution of selected individual events.

To quantify differences in elementary Ca²⁺ events between the cell lines a custom-written, automated algorithm was utilized to detect events and measure their amplitudes and durations (FIG. 4e). A striking difference between control and ASD lines was apparent in the numbers of detected sites, with control cells showing on average 97 sites per imaging field, whereas FXS and TS cells showed only 12 and 29 sites, respectively (FIG. 5a). The mean frequency of events per site appeared higher in control cells than in both FXS and TS cells (FIG. 5b), but quantification was imprecise because many sites, particularly in the FXS and TS cells, showed only a single event. Using the latency between the UV flash and first event at each site as an alternative measure of the probability of event initiation showed no significant difference among FXS, TS and control cell lines (FIG. 5c). Mean event amplitudes were also similar among the three cell lines (FIG. 5d). A second key difference between the control and FXS and TS cells was apparent in the durations of the local events. In all cell lines event durations were statistically distributed as single-exponentials, as expected for stochastic events. However, the time constants fitted to these distributions were appreciably shorter in FXS and TS cells as compared with control cells (FIG. 5e).

### Example 9: Conclusions

IP3-mediated Ca²⁺ signaling is a common phenotype and a shared functional defect in three distinct monogenic models of ASD and sporadic cases of ASD.

The implications of this work are:
GPCR-triggered intracellular Ca²⁺ release is decreased across several models of ASD;
TIRFM imaging determined that a striking difference between control and ASD lines arose in the numbers of detected sites and the durations of the local events;
IP3-mediated Ca²⁺ signaling may be a possible biomarker and a therapeutic target for ASD;
Alterations in Ca²⁺ homeostasis may be a common pathogenic mechanism in ASD, and may explain the heterogeneity of its symptoms.

### Example 10: Significance

Autism spectrum disorder (ASD) is a complex neurodevelopmental disorder affecting 2% of children. The socio-economic burden of ASD is enormous, currently estimated at over $268 billion per year in the USA alone. The rising rate of ASD, and the lack of drugs targeting its core symptoms, cry out for research into the development of new therapies. Drug development has proven to be problematic because of our limited understanding of the pathophysiology of ASD, the heterogeneity of symptoms, and difficulties in modeling the disease in vitro and in vivo. This is exemplified by the clinical failure of two large trials targeting the mGluR5 receptor as described in Mullard, Nature Reviews, Drug Discovery, 14 (2015) 151-53. Recent advances using monogenic animal models to understand the syndromic forms of ASD such as fragile X (FXS), Rett syndrome, and tuberous sclerosis (TS) have provided insights into the pathophysiology of these conditions. However, identified monogenic causes of ASD are responsible for only a few percent of all cases, with the majority caused by a complex interplay of various genetic and environmental factors.

Genome-wide association studies (GWAS) have identified many “risk” alleles for ASD, which cluster in common signaling pathways. This has led to a convergence hypothesis, proposing that key hubs within signaling pathways may be a point of convergence for many of the mutated genes to exert their deleterious effects. Recently, a GWAS of single nucleotide polymorphisms (SNPs) in over 30,000 cases revealed alterations in several Ca²⁺ channel genes associated with neurological disorders, including ASD, and other studies strongly implicated defects in Ca²⁺ channels and Ca²⁺-associated proteins with susceptibility to ASD. Taken together, Ca²⁺ signaling holds promise as a prospective ASD biomarker and therapeutic target.

The premise of this disclosure is based on the inventor's data showing robust deficits in inositol trisphosphate (IP3) mediated Ca²⁺ signaling in fibroblasts from a wide cohort of human subjects with both monogenic and sporadic ASD. In one embodiment, the inositol trisphosphate receptor (IP3R)—a Ca²⁺ channel within the endoplasmic reticulum (ER) organelle—is a signaling ‘hub’ where multiple ASD risk alleles and environmental factors interact to depress the normal functioning of intracellular Ca²⁺ signaling. Furthermore, this signaling defect highlights an intriguing and novel mechanistic link with deficiencies in mitochondrial bioenergetics. In one embodiment, the present disclosure provides improved Ca²⁺ signaling assays to investigate the prevalence of signaling abnormalities across monogenic and sporadic forms of ASD. In another embodiment, the present disclosure determines the molecular mechanisms underlying the defect. In one embodiment, the present disclosure elucidates how IP3R-mediated Ca²⁺ signaling deficits impact mitochondrial bioenergetics. In one embodiment, the present disclosure extend these studies to Ca²⁺ signaling in neurons derived from induced pluripotent stem cells (iPSCs) cells generated from fibroblasts from monogenic and sporadic ASD subjects.

### Example 11: Cell System Models for ASD

In one embodiment of this disclosure fibroblasts are utilized, which are readily obtained from skin biopsies and are already in routine clinical use for the diagnosis and development of therapeutic strategies of mitochondrial, peroxisomal and lysosomal organellar-based neurological diseases. The physiology of IP3 signaling in fibroblasts is well studied, which provided a validated and convenient model that complements the inventor's advanced imaging technologies to resolve IP3R functioning in intact cells at the single-molecule level. Moreover, fibroblasts are readily obtainable from both disease and matched control subject populations. In one embodiment, fibroblasts are employed as an amenable model system to refine the novel assays disclosed herein of IP3/Ca²⁺ signaling as a biomarker and potential diagnostic tool for ASD, and to investigate the molecular mechanisms underlying this shared defect and its downstream signaling consequences. In one embodiment, to then translate these findings to the disruptions in brain development and function thought to underlie autism, the inventors have extended their studies to neurons differentiated from iPSCs derived from the very same fibroblasts.

Although ASD is a complex heterogeneous disorder with poorly defined etiology, its high heritability suggests a strong genetic component. Capitalizing on the resources of the UCI Center for Autism Research and Translation (CART) fibroblasts are obtained from subjects with monogenic mutations associated with ASD (FXS, TS1, and TS2) and those with sporadic ASD lacking any such genetic defect. In one embodiment, the data and results disclosed herein demonstrate common defects in IP3R functioning among all these groups. FXS is the most common monogenic cause of ASD (˜5% of all cases), and is widely used as a model of ASD. It results from pathogenic expansion of a CGG-repeat on the X chromosome, leading to silencing of the Fragile X mental retardation (FMR1) gene and the absence of its corresponding protein, FMRP that binds mRNAs to regulate the translation of numerous proteins. Loss of FMRP leads to cognitive impairment and intracellular signaling defects. TSC is a syndrome caused by dominant mutations in one of two genes, TSC1 or TSC2, that produces ASD-like behaviors, seizures, intellectual disability and brain and skin lesions.

### Example 12: Ca²⁺ Signaling and its Disruption in Neurological Diseases

Ca²⁺ is a ubiquitous second messenger, participating in diverse cellular functions from excitability, motility, cell secretion and gene expression, to apoptosis. The spatial and temporal localization of Ca²⁺ signaling ensures high specificity of cellular responses. In neurons, IP3R-mediated Ca²⁺ release is involved in crucial functions including synaptic plasticity, memory, neuronal excitability, neurotransmitter release, axon growth and gene expression, highlighting the central integrating position played by IP3Rs.

The IP₃R is a Ca²⁺-permeable channel in the ER organelle membrane, regulating the release into the cytosol of Ca²⁺ sequestered within the ER. Channel opening requires binding of IP3, which is generated in response to activation of diverse cell surface receptors coupled through G protein or tyrosine kinase pathways. Moreover, the channel is biphasically gated by Ca²⁺; small elevations induce opening, whereas larger elevations cause inactivation. This property, together with the spatial distribution of IP3Rs results in a hierarchical organization of cellular Ca²⁺ signals (FIG. 8). Positive feedback by Ca²⁺ underlies regenerative Ca²⁺-induced Ca²⁺ release (CICR) that may remain restricted to a cluster of IP3Rs, producing local Ca²⁺ signals known as Ca²⁺ puffs, or may propagate throughout the cell as a saltatory wave by recruiting multiple puff sites by Ca²⁺ diffusion and CICR. Thus, IP3-mediated Ca²⁺ signaling represents a hierarchy of Ca²⁺ events of differing magnitudes, time course and spatial extent, and the clustered distribution of IP₃Rs is critical to proper cellular function (FIG. 8).

Disrupted functioning of ER Ca²⁺ release channels is observed in cognitive disorders including Alzheimer's and Huntington's diseases, and IP3Rs have recently been identified among the genes affected by rare de novo copy number variations in ASD patients. Moreover, the ER participates in a host of cellular responses to environmental stressors. Given that proper functioning of the IP₃R/Ca²⁺ signaling pathway is critical for normal neuronal development and function, in one embodiment, disruption of this pathway plays a key ‘hub’ role in the pathogenesis of ASD—one that serves as a diagnostic biomarker and target for novel drug discovery.

### Example 13: Energy-Deficient Endophenotype of ASD

How might disrupted IP₃R/Ca²⁺ signaling result in autistic phenotypes? In one embodiment, the inventors have proposed a novel link involving mitochondrial energetics. Biomarkers of mitochondrial energy deficiency are associated with a subset of ASD; a finding confirmed in ˜5% of ASD cases among a Portuguese population. A similar pattern of mitochondrial energy-deficiency is seen in syndromic ASD associated with Rett syndrome (RS) and in mouse models of RS; and the protein products of TSC1 and TSC2 regulate mTOR, a key regulator of mitochondrial function. Complementing these observations, Cardenas et al have described a direct role of constitutive Ca²⁺ release through IP3Rs in sustaining normal mitochondrial energetics, suppression of which leads to autophagy (C. Cardenas, et al, Essential regulation of cell bioenergetics by constitutive inositol trisphosphate receptor calcium transfer to mitochondria, Cell, 142 (2010) 270-283).

### Example 14: Ca2+ Signaling Through IP3Rs as a Pathophysiological ‘Hub’ in ASD

In one embodiment, the findings of Ca²⁺ signaling defects in monogenic and sporadic forms of ASD disclosed herein illustrates that the IP3R acts as a convergence hub where different forms of ASD intersect to exert their pathophysiological actions. The overall goal of the inventors were to determine the prevalence of IP3R dysfunction amongst ASD patients, to determine the molecular mechanisms underlying altered IP3R activity in human skin fibroblasts and iPSC-derived neurons from subjects with ASD, and to determine the downstream effects of altered IP₃/Ca²⁺ signaling on mitochondrial function. The significance and long-term impact of this disclosure lie in the following:
Multiple forms of ASD, both monogenic and sporadic converge to depress the normal functioning of the IP₃R/Ca²⁺ signaling pathway.
The finding that, in combination with behavioral testing, alterations in IP₃-mediated Ca²⁺ signals may aid in the diagnosis of ASD; an important advance given that early diagnosis and intervention are crucial for managing ASD, and that its extreme heterogeneity presently renders diagnosis challenging.
A mechanistic link between the long-recognized mitochondrial energy deficient endophenotype of ASD and this newly recognized molecularly defined Ca²⁺ signaling defect.
The discovery and understanding of fundamental Ca²⁺ signaling disruptions and their downstream consequences in ASD that are likely to inform potential therapeutic targets.

### Example 15: Biological Innovation

IP₃/Ca²⁺ signaling as a nexus in the pathophysiology of ASD. Microarray, whole-exome and whole-genome sequencing have identified over 800 loci with alleles contributing to ASD susceptibility. These are often found within the same signaling pathways, implicating an underlying genetic architecture of the disorder. In one embodiment of the disclosure herein, is a gene cluster often annotated as “synaptic function,” but which is clearly perceptible as “Ca²⁺signaling.” The quest for biomarker “signatures” of ASD is predominated by a search for genomic signatures, but to make this genomic information ‘actionable’, identification of functional markers is essential.

In one embodiment, the inventors have disclosed a role of Ca²⁺ signaling in the pathogenesis of ASD. Ca²⁺ release through IP3Rs at neuronal synapses underlies synaptic plasticity and memory, modulates neuronal excitability via Ca²⁺-activated K⁺ channels, and regulates dendritic pruning, neurotransmitter release, mitochondrial energetics and long term changes in gene transcription. In one embodiment, fibroblasts from patients with three distinct monogenic forms of ASD—FXS, TSC1 and TSC2—uniformly display depressed signaling through IP3Rs, and that a majority of those with sporadic ASD show the same defect. Thus, in one embodiment, the ER IP3R may serve as a ‘hub’ where multiple ASD risk alleles and environmental factors converge to confer their pathophysiological effect.

Ca²⁺ Signaling in Genome-Sequenced, Deeply-Phenotyped ASD Subjects.

To extend the inventor's studies of Ca²⁺ signaling defects to “typical ASD” subjects—in particular those displaying typical “sporadic ASD”—they capitalize on the unique resources of the UCI CART, such as, for example, stored skin fibroblasts, their iPSCs and derived neurons from a substantial cohort of subjects with ASD. Further, these patients have undergone deep behavioral phenotyping at CART. Phenotypes accumulated by CART include research grade ADOS (Autism Diagnostic Observation Schedule) evaluation, an Autism Diagnostic Interview (ADI), IQ evaluation, high density EEG monitoring under various conditions and complex sleep study evaluations and metabolomics (see Facilities). Using fibroblast cultures obtained from CART, the inventors have determined the prevalence of IP3/Ca²⁺ signaling defects across the autism spectrum and to statistically correlate these data with neurobehavioral phenotypes.

### Example 16: Technical Innovations

In one embodiment, the studies presented herein illustrate that the depressed global IP3-mediated Ca²⁺ signals in monogenic forms of ASD are mirrored by perturbations in local, subcellular ‘elementary’ signals. In one embodiment, this effect arises at the level of the IP3R channel itself, even though none of the monogenic models studied carries a mutation in the IP3R. It is thus a downstream point of signaling convergence. In one embodiment, these studies are performed at the single-molecule level, to determine how the ASD-linked disruptions are manifest in terms of the functioning and spatial distribution of individual IP3Rs. In one embodiment, the inventors determine how the gating and conductance properties of IP3Rs are modulated. In another embodiment, the inventors determine the spatial distribution of IP3Rs within the ER membrane—a crucial determinant for generation of local Ca²⁺ signals and Ca²⁺ waves through CICR. In another embodiment, the inventors determine the downstream consequences of altered Ca²⁺ signaling on cellular function. The innovative imaging approaches used in accomplishing these goals are further described herein.

Ca²⁺ Fluorescence Signals from Individual IP3Rs; the Optical Patch Clamp.

In one embodiment, the inventors have used the optical patch-clamp technique that allows imaging Ca²⁺ flux through single ion channels within intact cells with single channel resolution. Total internal reflection microscopy (TIRFM) (FIG. 9A) together with a slow Ca²⁺ buffer (FIG. 9B) was used to restrict excitation of a cytosolic fluorescent Ca²⁺ indicator to within ˜100 nm of the plasma membrane, thereby monitoring the local microdomain of elevated cytosolic [Ca²⁺] around the pore of Ca²⁺-permeable membrane channels. The resulting localized single-channel Ca²⁺ fluorescence transients (SCCaFTs) turn on and off rapidly, tracking channel openings and closings with a time resolution of a few ms (FIG. 9C). Using this technique the inventors dissected the Ca²⁺ puffs arising from clusters of IP3Rs (FIG. 9D) into the constituent openings and closings of individual receptor/channels (FIG. 9E).

Super-Resolution Imaging of Cellular Proteins.

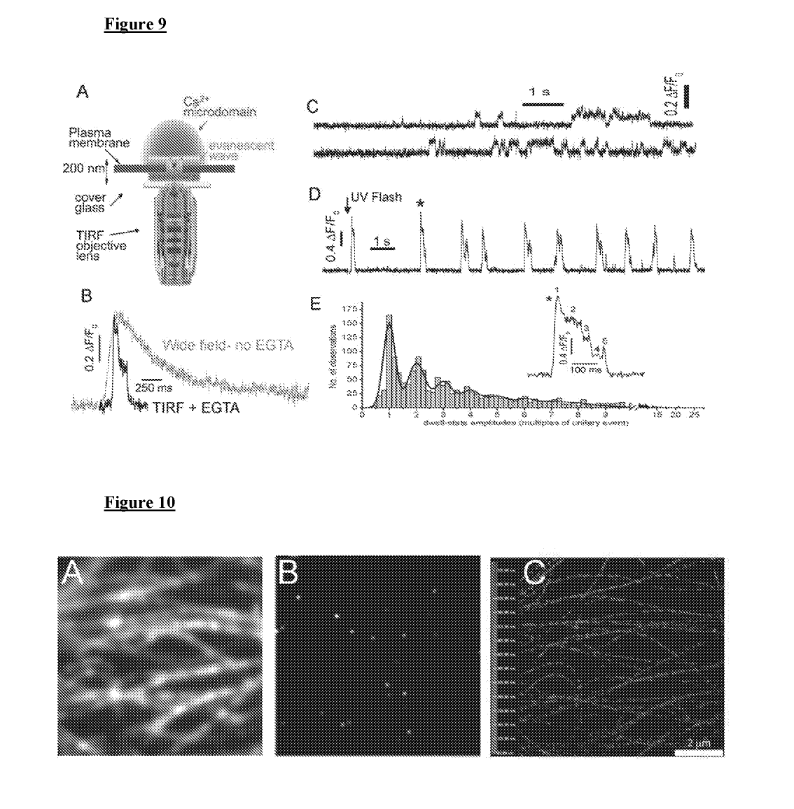

IP3Rs interact with one another via Ca²⁺ diffusion and CICR, and by allosteric coupling. Thus, defects in the generation and propagation of cellular Ca²⁺ signals may arise through changes in the spatial distribution of IP3R channels as well as through changes in their functioning. Given that IP3Rs are distributed as clusters of a few hundred nm diameter, they cannot be resolved by classical light microscopy. In one embodiment, the inventors utilized super-resolution imaging to side-step the diffraction limit, allowing the nanometer distribution of IP3Rs to be resolved. This process involved labeling a protein of interest (e.g. tubulin—see FIG. 10A) with an antibody conjugated to a photoswitchable dye or fluorescent protein to enable individual fluorophore molecules to be turned on and off in a sparse distribution. Their locations could then be determined with a precision of a few tens of nm using a Gaussian-fitting function (FIG. 10B). This process was repeated thousands of times until all molecules had been localized, generating a super-resolved image (FIG. 10C). The inventors have published super-resolved images of IP3R distribution within fixed cells (STORM), and visualized single IP3R molecules in live cells via overexpression of type 1 IP3R tagged with a photoswitchable fluorescent protein (PALM) in Smith et al, Single-molecule tracking of inositol trisphosphate receptors reveals different motilities and distributions, Biophys J, 107 (2014) 834-845.

### Example 17: Findings

IP3-Mediated Ca²⁺ Signaling is Depressed in FXS and TSCCC Fibroblasts.

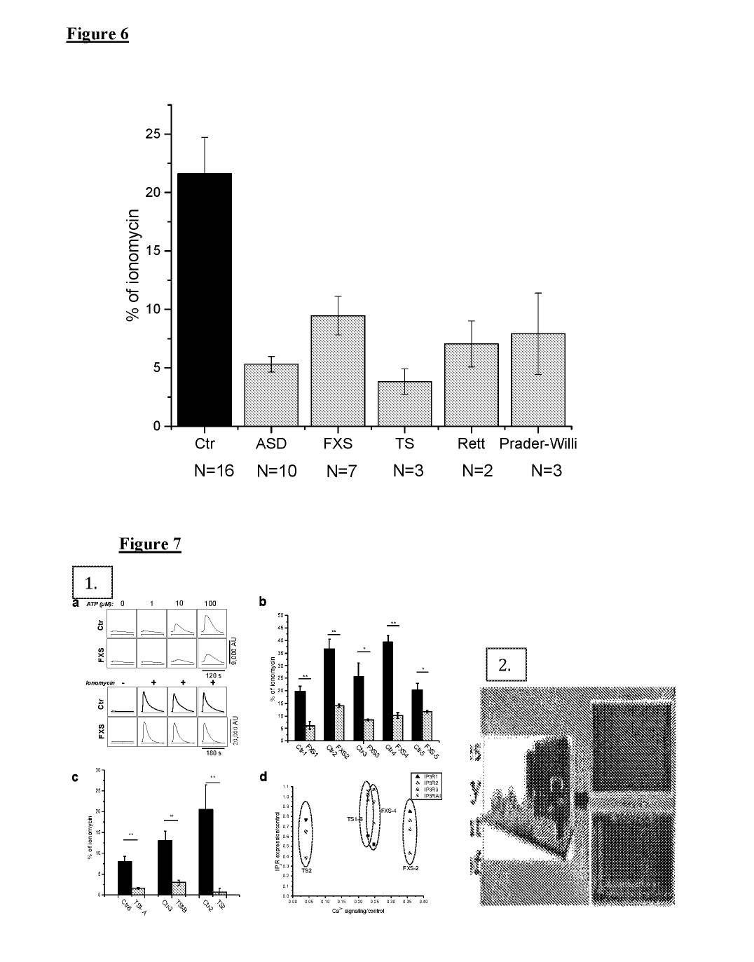

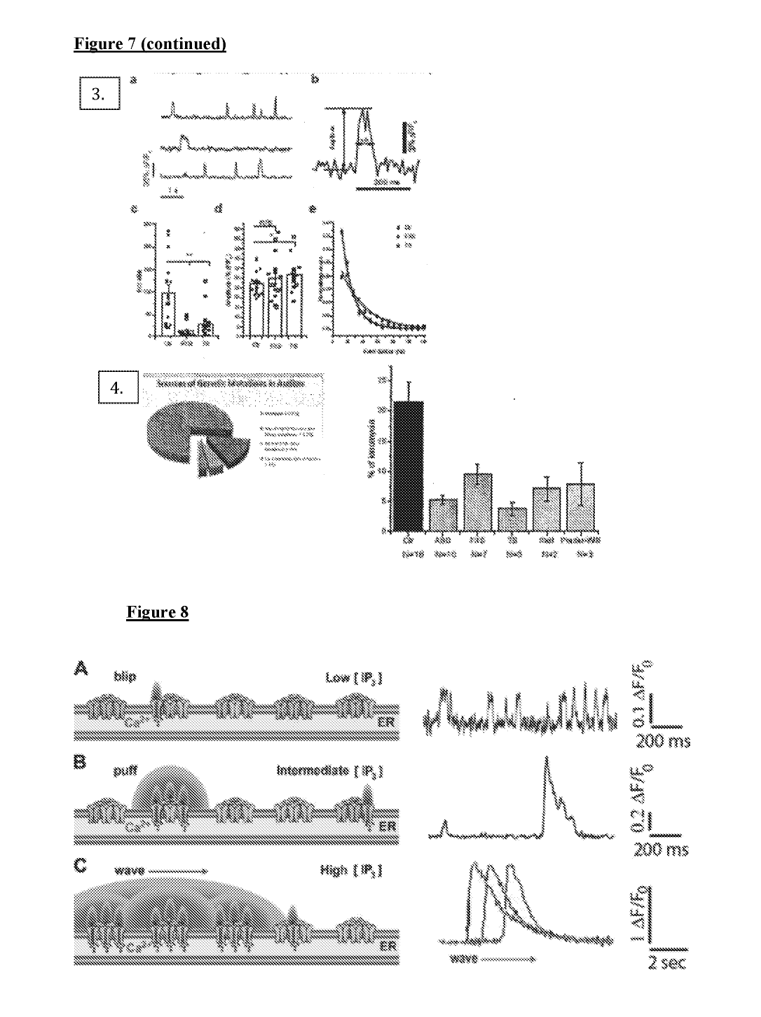

To look for defects in IP3-mediated signaling associated with ASD, a fluorometric imaging plate reader (FLIPR) was used to monitor cytosolic Ca²⁺ signals in skin fibroblasts from FXS and matched control subjects. ATP was applied to activate GPCR-linked purinergic P2Y receptors in Ca²⁺-free extracellular solution to exclude Ca²⁺ influx through plasmalemmal channels. Responses were significantly depressed in FXS cells (FIG. 7(1a), top; FIG. 7(1b)). This was not due to deficits in ER Ca²⁺ stores in FXS cells, as application of ionomycin in Ca²⁺-free media to completely liberate intracellular Ca²⁺ stores evoked similar signals in FXS and control cells (FIG. 7(1a), bottom). Cell lines from tuberous sclerosis (TSC1 and TSC2) patients further demonstrated deficits in ATP-evoked Ca²⁺ signals (FIG. 7(1c)), again without any appreciable difference in Ca²⁺ store content. Further, the diminished Ca²⁺ signals in FXS and TSC cells cannot be substantially attributed to diminished expression of IP3R proteins because IP3R expression showed little correlation with Ca²⁺ signaling depression (FIG. 7(1d)).

To then discriminate whether the observed deficits in ATP-induced signals in FXS and TSC cells arose through defects in GPCR-mediated generation of IP3, or at the level of IP3-mediated Ca²⁺ liberation, the GPCR pathway was circumvented by loading cells with caged IP3 (ci-IP3). Concordant with defects in ATP-induced Ca²⁺ signals, global cytosolic Ca²⁺ responses evoked by photoreleased i-IP3 in FXS cells were depressed and displayed slower kinetics. Corresponding measurements from TSC cells revealed even greater deficits in Ca²⁺ signal amplitudes.

IP₃-Signaling is Affected at the Level of Local Events.

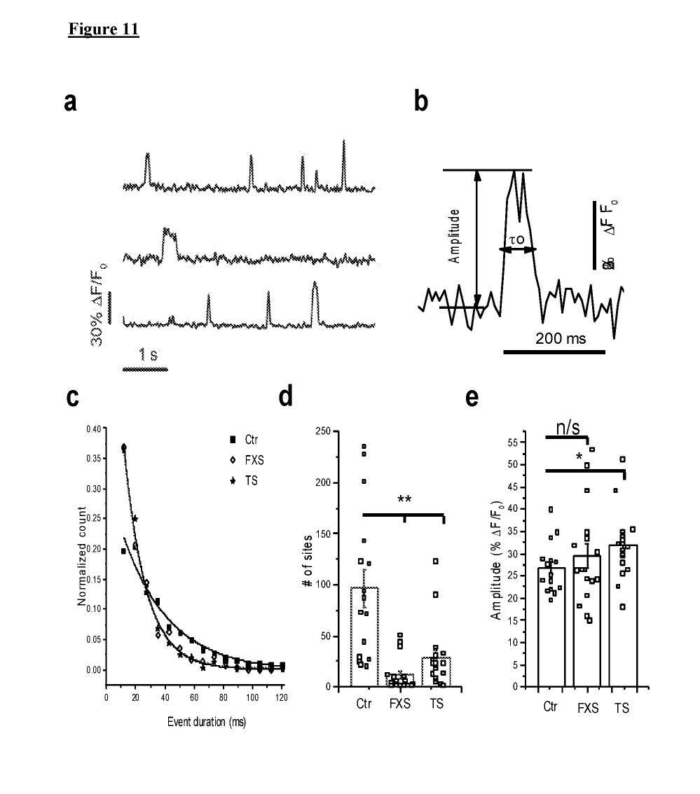

IP₃-mediated cellular Ca²⁺ signaling is organized as a hierarchy, wherein global, cell-wide signals arise by recruitment of local, ‘elementary’ events involving individual IP₃R or small numbers of IP₃Rs. These elementary events were imaged to elucidate how deficits in the global Ca²⁺ signals in FXS and TSC cells may arise at the level of local IP₃R clusters and individual channels. Ca²⁺ release evoked by spatially uniform photolysis of ci-IP₃ across the imaging field was apparent as localized fluorescent transients of varying amplitudes, arising at numerous discrete sites widely distributed across the cell soma (FIGS. 11a,b).

To quantify differences in elementary Ca²⁺ events between the cell lines a custom-written, automated algorithm was utilized to detect events and measure their durations, numbers and amplitudes. Local events were appreciably briefer in FXS and TSC cells (FIG. 11c), suggesting a shortening in mean open time of IP₃R channels. A second key difference was observed in the numbers of detected sites, which were strikingly different between control and ASD lines (FIG. 11d), although mean event amplitudes were similar (FIG. 11e).

Mitochondrial Energetics; a Putative Link Between Disrupted Ca²⁺ and ASD.

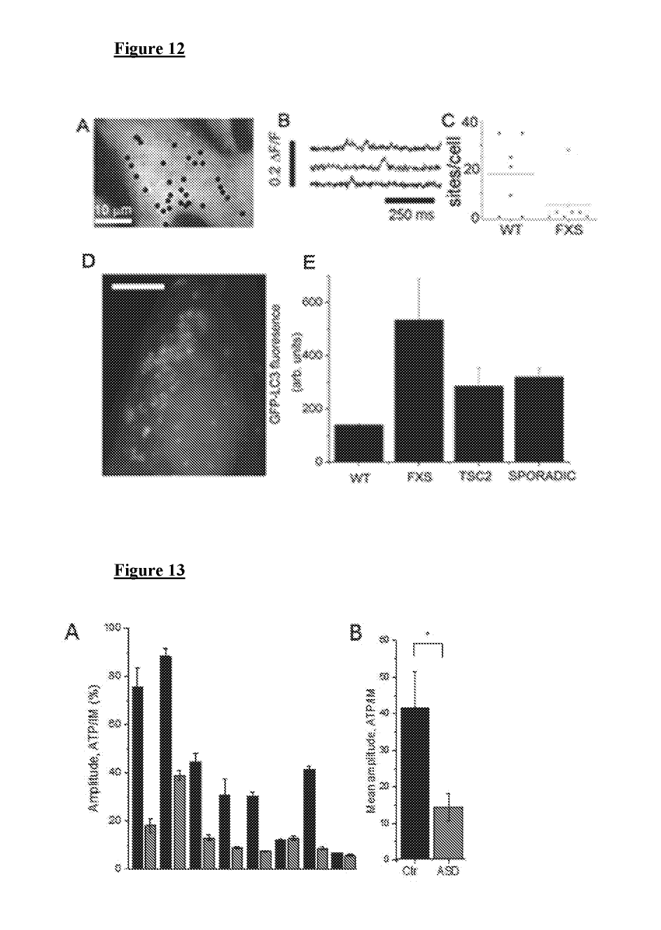

Low-level constitutive IP₃R-mediated transfer of Ca²⁺ from the ER to mitochondria maintains basal levels of oxidative phosphorylation and ATP production. In its absence, ATP levels fall, inducing AMPK-dependent, mTOR-independent autophagy. In light of mitochondrial energy deficient endophenotypes of autism, the inventors investigated whether constitutive Ca²⁺ signaling is impaired in ASD fibroblasts, leading to autophagy. Fibroblasts from FXS subjects displayed many fewer sites of local constitutive Ca²⁺ release than control cells (5+4 vs. 18+6 per cell), whereas signal amplitudes were similar. (FIG. 12A-C). To then investigate whether autophagy is upregulated in ASD, GFP-LC3 (a marker for autophagosomes) was expressed in fibroblasts from WT, FXS, TSC2 and a sporadic ASD subject recently enrolled in CART. GFP-LC3 fluorescence was significantly elevated in all ASD cases versus control (FIG. 12D, E). Significant elevations of lysotracker red fluorescence marking acidic lysosomes that bind autophagosomes were observed.

### Example 18: Experimental Design and Rigor

The experiments disclosed herein utilized facilities of the UCI CART; specifically fibroblasts and iPSC-derived neurons provided, respectively, by the Clinical and Stem Cell Cores. Cells are provided blind; identifiers from the CART RDP database are revealed only after the proposed experiments have been completed. Fibroblasts are obtained by skin biopsies from a large cohort of subjects exhibiting both monogenic and sporadic ASD. Subject numbers and statistical approaches are determined in consultation with biostatistician. ASD is a gender-biased disease affecting males with a frequency 4 times that of females. To correlate autism phenotypes with Ca²⁺ signaling abnormalities males and females are analyzed separately to avoid unknown confounding gender differences. Given the technically difficult and time intensive nature of subsequent single-cell mechanistic studies, limiting the number of independent lines studied, they are limited to fibroblasts and neurons from male subjects.

### Example 19: IP3R Signaling Defects as Endophenotypes of Monogenic and Sporadic ASD

In one embodiment, the inventors have found that IP3R dysfunction represents a hub where multiple forms of ASD, both sporadic and genetic, converge to produce an organellar neurological phenotype, analogous to the mechanism of mitochondrial encephalopathy. This finding is reinforced, and the etiological genetic web widened, by the data (FIG. 13) showing that fibroblasts from diverse “typical sporadic” ASD subjects display a defect in IP₃-mediated Ca²⁺ release, replicating previous published findings in monogenic forms of ASD. Fibroblasts have many advantages as a model cell system to study organellar phenotypes. Indeed, clinical evaluation and management of patients with mitochondrial encephalopathies has relied upon the use of fibroblasts for >25 years, despite the fact that neuronal dysfunction is central to their pathology and clinical phenotypes. In one embodiment, the inventors have optimized the FLIPR assay to enhance reproducibility and improve discrimination of Ca²⁺ signaling among sporadic ASD patients; and investigated correlations between Ca²⁺ signaling defects and neurobehavioral phenotypes and genotypes on an expanded cohort of ASD subjects. In one embodiment, the inventors have illustrated the utility of Ca²⁺ signaling as a biomarker and clinically-tractable discriminant for ASD susceptibility, and as a potential high-throughput screening tool for novel therapeutic agents.

Optimizing Reproducibility and Discrimination of the Ca²⁺ Signaling Assay.

In one embodiment, disclosed herein is a reproducible and reliable screening assay to quantify IP₃-mediated Ca²⁺ signals in biopsy-derived skin fibroblasts. Sources of variability arise in the culture of the cells and in the test assay itself. In one embodiment, the inventors have utilized confluent fibroblast monolayers in a FLIPR machine, recording peak Ca²⁺ signals in response to application of 100 μM ATP and expressing these relative to the peak response to the ionophore ionomycin to normalize for possible variation in ER Ca²⁺ store filling. Several factors in cell culture may introduce variability, including cell density, contact growth inhibition and position in cell cycle. Their influence on ATP and ionomycin responses was evaluated by comparing Ca²⁺ signals in a defined reference (unaffected) cell line across passage numbers (P10-P25), confluency levels (80% and 100% confluent), days after plating to achieve 100% confluency (2 and 4 days), and synchrony in cell cycle (by synchronizing to GO cell cycle arrest via 24 hr serum starvation). To investigate whether endogenous release of ATP by fibroblasts may depress responses to applied ATP by constitutively desensitizing purinergic receptors, wells were pretreated with apyrase (an ATP-hydrolyzing enzyme) that was washed out immediately prior to assay. Determining reproducible culture and assay conditions sets the stage for a robust and reproducible imaging protocol. Additional parameters are explored for their potential to enhance distinction between ASD and unaffected subjects, and between different endophenotypes of ASD. Existing and new data were examined for parameters including the kinetics of Ca²⁺ release, the rate of decay, and integral of the Ca²⁺ signals to determine whether, in conjunction with peak amplitude of the signal, they enhance correlation with a neurobehavioral phenotype.

IP₃ signaling in the FLIPR assay is activated by bath application of an agonist—in one example, ATP was used as an agonist to activate metabotropic purinergic receptors. This introduces complications and potential variability in the pathway leading to IP3 production. To circumvent that, a protocol is developed for delivering IP3 directly to the ER of permeabilized fibroblasts. This is based on established protocols utilizing a low-affinity fluorescent Ca²⁺ indicator (furaptra) trapped in the lumen of the ER and agents (e.g. saponin, streptolysin-O) to selectively permeabilize the cholesterol-rich plasma membrane, while sparing the cholesterol-poor ER. Moreover, this method enables controlling variability that may arise from intracellular factors (such as ATP concentration, cytosolic Ca²⁺ buffers, phosphatases and kinases) known to modulate IP₃R functioning.

Correlating Ca²⁺ Signaling Deficiencies with ASD Endophenotypes and Genotypes.

To study subjects exhibiting sporadic ASD, the inventors capitalize on resources of the UCI Center for Autism Research and Translation (CART), which is connected to the Autism Treatment Network-affiliated clinical autism center. Data and tissue samples are provided by genomics and clinical trials Cores. The CART “rapid discovery platform” database (RDP) includes rich phenotype and genotype information; the CART RDP database and samples from de-identified subjects were utilized.

In one embodiment, 300-400 study subjects are recruited during a time frame of 4 years (˜2 families/week for ˜100 ASD subjects/year). The functional profile of each subject is analyzed in the context of the Ca²⁺ signaling assay as optimized as disclosed above. Some subjects has a clinical diagnosis of autism and an ADOS score in the “autism” category, others may score in the milder “autism spectrum” category, and still others may fail to reach that diagnostic cut-off despite their clinical diagnosis (“non-spectrum”). In one embodiment, non-autistic normal siblings (who share half of the “risk” genome) and neurotypical controls whose family has no one with ASD or other neuropsychiatric diagnosis are included. In one embodiment, the inventors have disclosed to first establishing an association between autism diagnosis (using ADOS score cutoff) and the Ca²⁺ signal based on the FLIPR assay score, investigating differences in the mean signal score across quantiles of subjects (quartiles or smaller) and testing for a difference in the proportion of subjects with a diagnosis of autism using a chi-square test for trend.

In one embodiment, using the ADOS score as the gold standard for diagnosis, the inventors have disclosed constructing a Receiver Operator Curve (ROC) calculating sensitivity across the full range of values for the Ca²⁺ signal, choosing a cut-point for the signal that provides the best combination of sensitivity and specificity. Subsequently, using the Ca²⁺ signal information to define subsets of the subjects, differences are investigated between these subsets in phenotypic and genotypic characteristics from the RDP Database. The ability of the Ca²⁺ signal to discriminate ASD diagnosis from normal and strict autism using these same methods is also disclosed. Associations are carried out with this wealth of phenotype data in the RDP Database including age, gender, ethnicity, IQ (and sub-scores), ADOS (and sub-scores), EEG parameters (such as functional connectivity, diffuse slowing or cryptic seizures), sleep study parameters (including duration of sleep, latency to sleep or interruption of sleep), and metabolomics (e.g. lactate/pyruvate, carnitine, volatile metabolite). The presence and absence of inherited and de novo CNVs and variants in CART-targeted Ca²⁺ signaling loci are scored. Statistical methods include analysis of variance to examine differences in continuously measured variables between groups (such as quantitative test scores), and chi-square tests for categorical variables (such as diagnostic categories or presence and absence of seizures). In addition, multivariate methods, including logistic regression methods, are used to explore associations between multiple independent predictors of autism diagnosis. Approximately 70% of subjects meet full criteria based on ADOS and Ca²⁺ signal properties while 15% are classified “typical normal” and the remaining 15% are intermediate. With a sample size including approximately 400 subjects (subgroups including 280, 60 and 60 subjects), there is 80% power to detect an effect size equal to 0.15 SD, a relatively small difference. Analyses are considered exploratory, thus no adjustment for multiple comparisons will be made. Therefore there is ample power to test the utility of FLIPR Ca²⁺ signal in the diagnosis and endophenotyping of ASD.

In summary, in one embodiment, the inventors have determined how well the Ca²⁺ signal correlates with the autism phenotype and in particular ascertain whether scores associate with subgroups of standard autism characteristics or features of ASD that is uncovered in the RDP database. The Ca²⁺ signal is a good biomarker for ASD or a subset of this disorder, and these results provide a proof-of-concept foundation for a double-blinded clinical trial of IP₃/Ca²⁺ signaling as a diagnostic and for screening for potential novel therapeutics.

### Example 20: Molecular Mechanisms Underlying Disrupted IP₃-Mediated Ca²⁺ Signaling in ASD

Elucidation of the mechanisms responsible for the decreased IP3-mediated Ca2+ response in ASD is essential for the rational development of screening and treatment strategies. The results disclosed herein illustrate that Ca²⁺ signaling defects in FXS, TSC and sporadic ASD primarily arise downstream of GPCR-mediated IP₃ production, at the level of Ca²⁺ liberation through IP₃Rs. Given that the Ca²⁺ store content of the ER is unaffected, either or both of two distinctly different mechanisms may be involved: (i) Alterations in the spatial patterning of IP3Rs which affect their coordination by Ca²⁺ diffusion and CICR. (ii) Disruptions in the functional gating properties of individual IP3R channels. Recent advances in imaging technology (the ‘optical patch clamp’ and super-resolution microscopy) enable the investigation of these topics at the single-molecule level in intact cells. Further, alterations in cAMP-modulation of IP3Rs may underlie the depressed Ca²⁺ signals. For all these mechanistic studies fibroblasts are employed as a tractable model cell system for organellar disease, which allows investigation of cells from sporadic ASD patients in addition to those with known monogenic disease.

ASD-Linked Deficits in Ca²⁺ Signaling Result from Altered Spatial Distribution of IP3 Receptors.

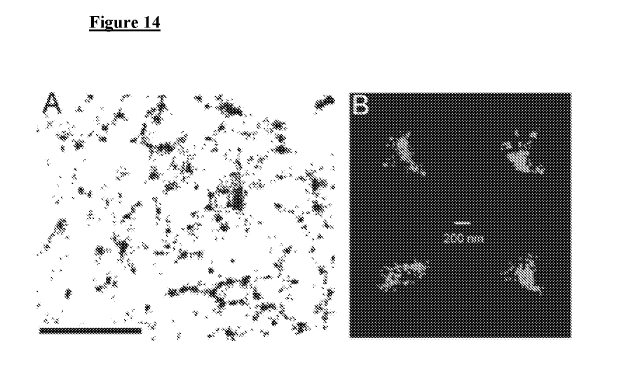

The spatial distribution of IP3Rs is crucial for determining the patterning of IP3-induced Ca²⁺ release, affecting both local and global Ca²⁺ signals. This arises because IP3Rs display Ca²⁺ induced Ca²⁺ release (CICR), meaning that Ca²⁺ released from one IP3R will diffuse and potentiate the release of Ca²⁺ from closely neighboring IP3Rs. The inventor's data demonstrate a consistent reduction in ATP-evoked Ca²⁺ release at the whole-cell level in FXS and TS fibroblasts (FIG. 7(1b, 1c); FIG. 11) and in numbers and kinetics of local Ca²⁺ events (FIG. 12). These observations cannot be attributed simply to reduced expression of IP₃Rs (FIG. 7(1d)), but is likely to result if the clustered distribution of IP₃Rs (FIG. 14) were disrupted, thereby increasing the separation between IP₃Rs and hindering diffusion of Ca²⁺ between them. Because the dimensions of IP₃R clusters are smaller than the diffraction limit of classical light microscopy, Nikon STORM super-resolution microscope is used to resolve the spatial patterning of IP₃R distribution at a scale of tens of nanometers. IP₃R distribution is mapped by super-resolution STORM immunofluorescence in fixed cells, using well-characterized antibodies specific for the type 1, 2 and 3 IP₃R isoforms to investigate the nanoscale organization of IP₃Rs (FIG. 14). The spatial patterning of control cells are determined to illustrate that different isotypes display different propensities to cluster, number of IP₃Rs in a cluster, dimensions of a cluster, and distance between the clusters. These parameters powerfully influence the amplitude and frequency of Ca²⁺ puffs generated at individual clusters, and the ability of Ca²⁺ waves to propagate from cluster to cluster. The differences between control and FXS and TSC cells underlie the defects in functional Ca²⁺ signaling. For example, the observed reduction in numbers of puff sites in FXS cells (FIG. 11d) might result if there were fewer clusters of IP₃Rs, even if the total numbers of receptors were unchanged.

Single-Channel Properties of IP₃Rs in FXS, TSC, and Control Fibroblasts.

The optical patch clamp technique was utilized to resolve the activity of individual and clustered IP3Rs and determine how their gating and Ca²⁺-permeation properties are affected in FXS and TSC. This technique enables resolving single-channel openings as well as puffs comprised of several concerted channel openings that are evoked following photolysis of caged IP3. Important factors in this process are the opening probability of each channel, amount of Ca²⁺ released per elementary event (Ca²⁺ permeability and mean channel open time), and the number of functional channels per cluster.

In one embodiment, the techniques disclosed herein are useful for enhanced TIRF imaging and automated detection and analysis of local Ca²⁺ signals to quantify the numbers and distribution of functional receptor sites participating in local Ca²⁺ responses in FXS, TSC and control cells, and estimate the number and dynamics of channels participating in each release event. The single channel (“blip”) amplitude and duration was assessed, serving as a measure of Ca²⁺ channel permeability and mean channel open time. The frequency of events and latency between the UV flash and the first event at each site are used as measures of the probability of channel opening. The event duration as a function of the channel gating kinetics was also measured. Importantly the step-wise transitions in fluorescence levels was imaged on the falling phase of puff events that represent the closings of individual IP₃R channels, which allow counting the number of functional channels that contribute to local Ca₂₊ events. By measuring amplitude of each event and step-wise closings, the number of participating channels in each cluster was estimated, and measured the open probability and open time of each channel in ASD cells and control cells.

Restoration of Ca²⁺ Signaling by Cyclic AMP.

The second messenger cAMP regulates many neuronal processes, including mnemonic processing and anxiety, which are associated with FXS. cAMP-dependent protein kinase A (PKA) phosphorylates the IP3R and potentiate its activity. PKA is a target of FMRP and lowered levels of cAMP have been shown in both drosophila and mouse models of FXS, suggesting that altered levels of cAMP play a mechanistic role in defective IP3-mediated Ca²⁺ signals.

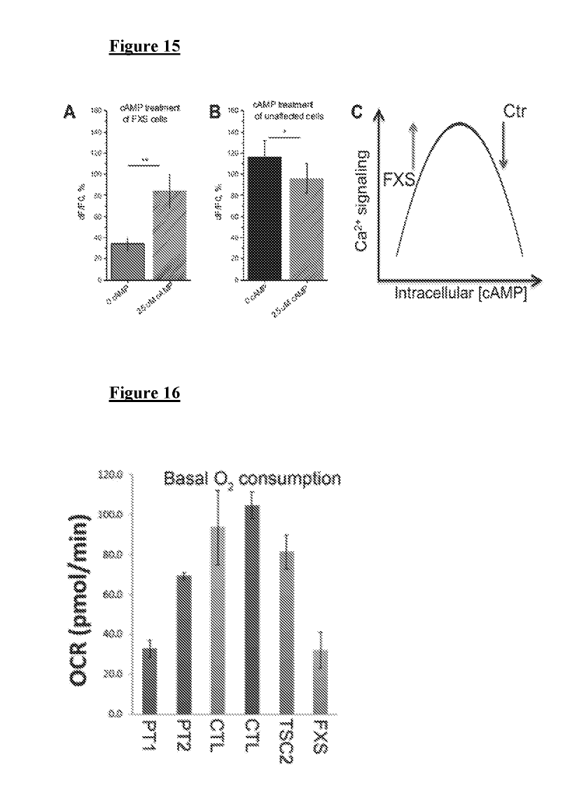

In one embodiment, the results disclosed herein illustrate that a cell membrane-permeant cAMP analog (8-bromo-cAMP) substantially rescued the depressed global Ca²⁺ response to photorelease of IP₃ in FXS fibroblasts. (FIG. 15A). Strikingly, cAMP had the opposite effect on control cells, lowering the peak amplitude (FIG. 15B). This effect may be due to a biphasic action of cAMP on Ca²⁺ signaling, resulting in an inverted U shaped dose-response relationship. In one embodiment, cAMP levels are lowered in FXS cells, so that addition of exogenous cAMP potentiates IP3-mediated Ca²⁺ release, whereas further increases beyond the higher basal cAMP concentrations in control cells may depress IP₃R function (FIG. 15C).

Basal levels of endogenous cAMP in control, FXS and TSC fibroblasts were measured by commercial immunoassays. Next, concentration-dependence curves are derived for IP3-evoked Ca²⁺ signals in fibroblasts. If the deficit in FXS arises because of a lower basal level of cAMP, pretreatment of cells with adenyl cyclase inhibitor to reduce basal cAMP levels diminishes Ca²⁺ signals in control cells to the level in FXS cells. Pharmacological approaches were explored to elevate cAMP levels by inhibiting phosphodiesterase enzymes. Of particular interest, the phosphodiesterase inhibitor Rolipram is an FDA-approved antidepressant that has shown promise for psychotic conditions and has potential to treat certain forms of ASD.

Ca²⁺ Signaling in Fibroblasts from Sporadic ASD Subjects.

Fibroblasts from 3 sporadic ASD subjects were selected that show large deficits in Ca²⁺ signaling and their matched controls. These cells are examined to determine whether these cells display alterations in IP3/Ca²⁺ signaling at the single channel level that mimic the findings obtained for monogenic forms of ASD.

### Example 21: Constitutive Ca²⁺ Signaling, Mitochondrial Energetics and Autophagy in ASD

Constitutive IP3R-mediated transfer of Ca²⁺ from ER to mitochondria maintains normal mitochondrial function—mitochondria need to be ‘fed’ Ca²⁺. In its absence, cells undergo an energy crisis; oxidative phosphorylation is compromised, ATP levels fall and AMPK-dependent, mTOR (mammalian target of rapamycin) independent autophagy is induced as a mechanism to enable cells to survive. Reduced mitochondrial activity is reported across monogenic and sporadic ASD; autophagy is important in neurodevelopment; and developmental alterations of excitatory synapses in ASD are attributed to defective regulation of autophagy. In one embodiment, the inventors have investigated a novel putative link, whereby disrupted IP3R signaling contributes to the pathogenesis of ASD. IP3-evoked Ca²⁺ signals are transmitted to mitochondria to enhance function, and constitutive uptake of Ca²⁺ was identified through IP3Rs in the absence of overt stimulation as required to maintain basal mitochondrial bioenergetics.

In one embodiment, the inventors have found that a reduction in constitutive IP3-mediated Ca²⁺ release events disrupts normal mitochondrial bioenergetics, creating the “energy deficient endophenotype of ASD.” This is illustrated in FIG. 16, showing reduced constitutive Ca²⁺ activity and depressed basal mitochondrial metabolism in fibroblasts from ASD patients (FIG. 16).

Depressed Constitutive Ca²⁺ Signaling in ASD.

In one embodiment, the inventors have disclosed depressed constitutive Ca²⁺ signaling in FXS fibroblasts (FIG. 12). In one embodiment, this arises directly from the same mechanisms that depress IP3-evoked signals. In one embodiment, both are reduced to comparable extents in FXS vs. control fibroblasts. In one embodiment, sites of evoked and constitutive Ca²⁺ release map onto one another. In one embodiment, the reduced occurrence of constitutive Ca²⁺ signals in FXS arises from reduced basal IP3 levels. In one embodiment, this deficit in constitutive Ca²⁺ signaling is restored by over-expression of IP3R isoforms, or elevation of basal [IP3] (by sustained ‘tickling’ with low concentration of ATP, or by modulating cAMP levels. In one embodiment, these observations are extended to fibroblast from TSC and selected sporadic ASD patients to determine whether this is a common defect across multiple forms of ASD and the extent to which depressed constitutive IP3R signaling correlates with the severity of autism.

Ca²⁺ Signaling and Mitochondrial Energetics.

The causal, downstream effects of depressed constitutive Ca²⁺ signaling on mitochondrial energetics were examined. The overall mitochondrial bioenergetics were evaluated using the Seahorse XF (Agilent Technologies) and commercially available “cell-mito” and “glycol” stress-test kits; high-throughput protocols that provide direct and detailed information on mitochondrial respiration and glycolysis. Basal oxygen consumption rate (OCR), a measure of mitochondrial respiration and extracellular acidification rate (ECAR), a measure of glycolytic activity are taken before sequential addition of mitochondrial inhibitors oligomycin, FCCP and rotenone+antimycin, allowing calculation of basal, maximal and reserve respiratory rate, ATP production and proton leak. These parameters are determined in fibroblasts from monogenic and sporadic ASD subjects, and relate them to constitutive Ca²⁺ signaling in the same cell lines.

Utilizing selected fibroblast lines showing large deficits in mitochondrial function associated with Ca²⁺ signaling defects, the mechanism of IP3/Ca²⁺ calcium signaling impacting mitochondrial energetics was defined. The mitochondrial respiratory chain is composed of five complexes, each encoded by many genes. Patients with mitochondrial disease are routinely screened to determine which respiratory complex is affected, yielding a more specific diagnosis, revealing potential mutated gene candidates and suggesting potential cofactor therapeutics. This approach was applied to ASD, utilizing the Seahorse XF and their “plasma membrane permeablizer” kit and technical protocol for measuring respiratory complexes without mitochondrial purification, exploiting the fact that substrates feed differentially into mitochondrial pathways to isolate which respiratory complex(es) are responsible for the altered OCR in the selected fibroblast lines. In one embodiment the inventors determined whether a single complex is the primary target, as is usually the case in primary mitochondrial disease. In another embodiment, the inventors determined that it is simply bulk NADH-reducing equivalents that result from limiting PDH activity. Citrate synthase levels provide a measure of mitochondrial numbers. The inventors determined whether mitochondrial numbers (assessed by citrate synthase levels) are altered, and whether depressed Ca²⁺ signaling correlate with increased production of the reactive oxygen species that are implicated in ASD. These experimental results precisely reveal the site at which the control and ASD lines differ in mitochondrial energetics and better highlight targets for therapeutic intervention.

Autophagy as a Downstream Consequence of Disrupted Ca²⁺ Signaling and Mitochondrial Energetics in ASD.

The induction of autophagy consequent to mitochondrial energy deficiency is implicated in ASD. In one embodiment, the inventors extended their initial studies showing increased autophagy in FXS cells (FIG. 12) to other monogenic cell lines (TSC 1 & 2), and to cells from sporadic ASD subjects and their matched controls, utilizing fluorescence of lysotracker red or GFP-LC3 as monitors of autophagy. To test the causality between depressed constitutive IP3R signaling and the induction of autophagy rescue experiments are performed. In one embodiment, the induction of autophagy can be suppressed by restoring constitutive Ca²⁺ signaling as described in this disclosure.

Autophagy is canonically regulated by the mTOR dependent signaling pathway, and GWAS studies have identified many candidates on this pathway as associated with ASD. Distinct from this, a novel role for IP3/Ca²⁺ regulation of autophagy in an mTOR independent fashion is known to one skilled in the art. This arises from decreased mitochondrial-ER crosstalk of Ca²⁺ resulting in hyperphosphorylated pyruvate dehydrogenase (P-PDH), limiting the supply of reducing equivalents to the citric acid cycle (TCA) and consequent reduction of ATP production. The rising AMP:ATP ratio then phosphorylates AMPK to induce pro-survival autophagy, and genetic mutations resulting in decreased PDH activity are associated with ASD phenotypes. In one embodiment, the inventors investigate whether this mTOR-independent pathway links reduced constitutive IP3 signaling to induction of autophagy in ASD fibroblasts. Levels of P-PDH and P-AMPK are elevated in ASD versus control; this is determined by western blot. Furthermore, levels of P-PDH (using a PDH kinase inhibitor dichloroacetic acid) and P-AMPK (using Compound C, AMPK inhibitor) are manipulated to determine whether there is an associated drop in autophagy levels. In one embodiment, the inventors overcame the deficit in reducing equivalents arising from a lack of IP3 signaling by enhancing the availability of TCA cycle intermediates to suppress induction of autophagy.

### Example 22: From Skin Fibroblasts to iPSC-Derived Neurons: A Model Cell System to Study ASD

Although human skin fibroblasts have advantages as a model cell system to study ASD, the central pathology of ASD lies in neuronal dysfunction. To truly understand the disease pathogenesis, examination of IP3/Ca²⁺ signaling in neurons is needed. Animal models for neuronal diseases, such as transgenic mice, provide only an imperfect system, exemplified by the recent failure of two clinical ASD drug trials, and >100 failed clinical trials of Alzheimer's disease therapies, highlighting the hazards of translating research findings from mice to humans. However, recent advances in stem cell biology together with the advent of somatic cell reprogramming now enable the generation of patient-derived induced pluripotent stem cells (iPSCs) that can be differentiated in vitro into neurons, glia and other cell types. It is only very recently that this technology has allowed for the study of neuronal disease phenotypes, such as the long-studied mitochondrial encephalopathies, in neurons. In one embodiment, the inventors have extended their studies in ASD to human iPSC-derived neurons. In one embodiment, the inventors have determined how ER IP3R/Ca²⁺ signaling may be altered in neuronal cells from ASD subjects, how these alterations relate to the corresponding deficits in the fibroblasts from which the cells are derived, and to begin to explore consequences for neuronal function.

Differentiation of iPSC Cell Lines.

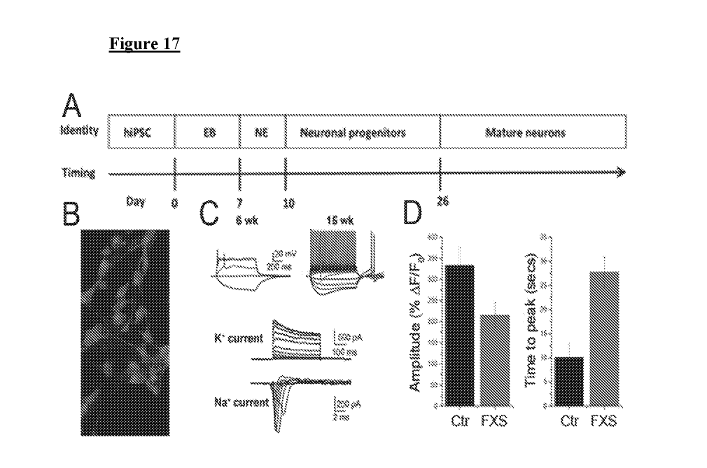

In one embodiment, the inventors have determined the extent to which the IP3-mediated Ca²⁺ signaling abnormalities observed in fibroblasts from ASD patients are evident in neurons derived from these same fibroblasts. In one embodiment, GABAergic interneurons—a neuronal cell type strongly implicated in ASD—were derived from CART ASD skin fibroblasts (FIG. 17). These provide the iPSC derived neurons used herein. Six ASD lines in total are studied (TSC1, TSC2 and FXS plus three sporadic ASD cases); each line is paired with a closely matched neurotypical control. FIG. 17D provides evidence of disrupted IP3-mediated Ca²⁺ signaling in neuronal progenitors from an FXS subject, indicating the signaling abnormality observed in fibroblasts is conserved through the derivation process.

IP3-Mediated Ca²⁺ Signaling in iPSC-Differentiated Neurons.

GABA interneurons are identified using lentiviral-mediated delivery of a vesicular GABA transporter (VGAT) promoter driven fluorescent construct (pLV-hVGAT-mCherry). Cytosolic Ca²⁺ is imaged with Cal-520 to achieve spectral separation from the mCherry signal. Experiments are performed in the presence of tetrodotoxin to suppress spontaneous action potentials that would otherwise complicate interpretation by evoking voltage-dependent and synaptically-mediated Ca²⁺ influx. IP3-mediated Ca²⁺ liberation are evoked by group 1 metabotropic receptor agonists (DHPG and ACPD) delivered either by bath application, or by photorelease from caged precursors to enable local application to selected regions of soma or dendrites via a focused UV laser spot. To bypass upstream elements in the signaling pathway neurons are loaded with caged IP3, to directly and specifically activate IP3Rs by localized photo-uncaging. Multiple recordings are obtained from each imaging dish after which cells will be fixed and stained with well-characterized antibodies to discriminate different subtypes of GABA interneurons (parvalbumin, calretinin, somatostatin or calbindin).

In one embodiment, the inventors compare IP3-mediated Ca²⁺ signals in neurons obtained from control, FXS, TS1 and TS2 subjects. In light of the results disclosed herein and the ubiquity of the IP3/Ca²⁺ signaling pathway across all cells of the body, it is likely that the deficits observed in fibroblasts from ASD subjects are reflected in neuronal function. In one embodiment, paired comparisons are made of somatic Ca²⁺ signals in fibroblasts and neurons obtained from the same subjects. Essentially, a scatter plot was derived in which each data point represent the ratio of Ca²⁺ signal in neurons vs. fibroblasts for each patient. This reveals the extent to which neuronal deficits can be inferred from measurements in fibroblasts, and whether similar relationships hold across different monogenic and sporadic cases of ASD. In one embodiment, the inventors find a qualitative agreement with their findings in fibroblasts. This strengthens the view that Ca²⁺ signaling deficiencies play a causative role in ASD pathogenesis, and begins to reveal that these defects are specific to particular neuronal subtypes.

### Example 23: Ca²⁺ Signaling

Disease of the intracellular organelles is a rapidly emerging area of medicine with several Mendelian genetic prototypes already well recognized to produce distinctive spectra of diseases of the mitochondria, the lysosomes and the peroxisomes, and extensions of these disease mechanisms now beginning to advance our understanding of common complex polygenic disorders having a shared organellar pathophysiology. Unlike the other organelles, the endoplasmic reticulum (ER) lacks a well-defined spectrum of genetic diseases. In one embodiment, the disclosure here illustrates that rare ataxia syndromes may be caused by mutations of the ER inositol 1,4,5-trisphosphate receptors (IP₃R). In some embodiments, the organelle may play a role in complex polygenic syndromes (such as Alzheimer's disease) by altering the function of ER Ca²⁺ channels. Moreover the organelle may play a role in a host of cellular responses to environmental stressors. Organelles are expressed in essentially all cells of the body but overwhelmingly organelle disease is manifest in the central nervous system (CNS). On the other hand, organelle function is typically studied in human fibroblasts and such skin biopsies are already in routine clinical use for the functional diagnosis of mitochondrial, peroxisomal and lysosomal neurological disease. Since IP₃R function in signaling has been extensively studied in fibroblasts, functional diagnostics for the ER neurological diseases are rendered similarly feasible.

In one embodiment of the present disclosure, multiple genetic lesions may lead to autism spectrum disorder (ASD), a common complex polygenic disorder characterized by difficulties in social interaction, communication and restricted, repetitive behavior, converge to perturb normal Ca²⁺ signaling, and that depressed function of the ER IP₃R in the Ca²⁺ signaling pathway plays a key ‘hub’ role in the pathogenesis of ASD—one that might serve as a diagnostic biomarker and potential target for novel drug discovery. The IP₃R is a Ca²⁺-permeable channel in the ER organelle membrane, regulating the release into the cytosol of Ca²⁺ sequestered within the ER. Channel opening requires binding of IP₃, which is generated in response to activation of diverse cell surface receptors coupled through G protein or tyrosine kinase pathways. Moreover, the channel is biphasically gated by Ca²⁺; small elevations induce opening, whereas larger elevations cause inactivation. This property, together with the spatial distribution of IP₃Rs results in a hierarchical organization of cellular Ca²⁺ signals. Positive feedback by Ca²⁺ underlies regenerative Ca²⁺-induced Ca²⁺ release (CICR) that may remain restricted to a cluster of IP₃Rs, producing local Ca²⁺ signals known as Ca²⁺ puffs, or may propagate throughout the cell as a saltatory wave by recruiting multiple puff sites by Ca²⁺ diffusion and CICR. Thus, IP₃-mediated Ca²⁺ signaling represents a hierarchy of Ca²⁺ events of differing magnitudes, time course and spatial extent, and the clustered distribution of IP₃Rs is critical to proper cellular function.

In one embodiment, the inventors have demonstrated depressed IP₃-mediated Ca²⁺ signaling as a shared feature in three distinct monogenic syndromes highly comorbid with autism spectrum disorder (ASD)—fragile X syndrome (FXS) and tuberous sclerosis syndrome type 1 and type 2 (TSC1 and TSC2). A fluorometric imaging plate reader (FLIPR) was used to monitor, in a Ca²⁺-free extracellular solution, cytosolic Ca²⁺ signals induced by ATP activating GPCR-linked purinergic P2Y receptors in skin fibroblasts from matched affected and control subjects. Responses were significantly depressed in cells from all three monogenic syndromes, and this was not due to deficits in ER Ca²⁺ stores, nor due to diminished expression of IP₃R proteins. To discriminate whether the observed deficits in these monogenic models of ASD arose through defects in GPCR-mediated generation of IP₃, or at the level of IP₃-mediated Ca²⁺ liberation, the inventors circumvented the GPCR pathway by loading cells with caged IP₃ (ci-IP₃), and observed similar defects in global cytosolic Ca²⁺ responses evoked by photoreleased i-IP₃. Since IP₃-mediated cellular Ca²⁺ signaling is organized as a hierarchy, wherein global, cell-wide signals arise by recruitment of local, ‘elementary’ events involving individual (or small clusters of) IP₃Rs, an optical patch clamp technique was utilized to image these elementary events and molecularly elucidate how deficits in the global Ca²⁺ signals in these diverse monogenic ASD model cells arise at the level of local IP₃R clusters and individual channels. Ca²⁺ release evoked by spatially uniform photolysis of ci-IP₃ across the imaging field was apparent as localized fluorescent transients of varying amplitudes, arising at numerous discrete sites widely distributed across the cell soma. To quantify differences in these elementary Ca²⁺ events between the cell lines, the duration, number and amplitude of these quantal local events were measured. A dramatic shortening of the mean open time was observed—a flicker opening lasting about one-half as long as the control—for all the ASD model cells' IP₃R channels, and an apparent decrease in the numbers of definable release sites. However, the latency to first opening and the mean event amplitudes were similar in all cells. These results illustrated that the IP₃Rs, carrying no mutations themselves, are functionally altered at the level of single (or small clusters of) channels in these three distinct ASD models.

ASD symptoms and their severity vary widely across autistic individuals, making it a challenge to diagnose this complex spectrum encompassing many phenotypes and co-morbidities, and giving rise to a tragic “diagnostic odyssey” that delays diagnosis, and hence treatment, until the typical mean age ranging from 2 years to 5 years. The diagnosis of ASD is made based on questionnaires and behavioral tests, relying on parent observations and comprehensive evaluation by psychologists, pediatricians, psychiatrists, and speech therapists. Current lack of biomarkers and molecular targets makes diagnosing, studying and treating autism a challenging task. Moreover, early diagnosis of autism before manifestation of behavioral symptoms is critical for optimal intervention, and accurate diagnosis is crucial in order to exclude other potential conditions which may require different types of therapies.

Recent advances in genetics have greatly improved understanding of the pathophysiology of autism, identifying a handful of monogenic syndromes but also over 800 genes contributing to susceptibility for autism and thereby providing genetic models for studying this condition. These findings imply that although one highly penetrant mutation is enough to cause ASD, this is very rare, and that the number of the identified genes with a potential to contribute to the majority of cases is too large to be of utility for diagnosis. This is the case since, while highly heritable, the polygenic pattern of inheritance ASD follows implies that there are several highly heterogeneous weakly penetrant genetic variants, either arising de novo or inherited from parents that, in combination with environmental risk factors, cause ASD. In view of this, the inventors have studied single genes and monogenic disorders, such as fragile X and tuberous sclerosis, while also appreciating how many susceptibility factors participate in a common functional pathway, such as excitation/inhibition, synaptic transmission, or Ca²⁺ homeostasis, that may be a point of convergence for many of the mutated genes to exert their deleterious effects. Ca²⁺ signaling is a potential root defect, since it is a ubiquitous second messenger, participating in diverse cellular functions from neuronal excitability, neurotransmitter release, cell secretion and gene expression, to apoptosis. The spatial and temporal localization of Ca²⁺ signaling ensures high specificity of cellular responses. In neurons, IP₃R-mediated Ca²⁺ release is involved in crucial functions including synaptic plasticity, memory, neuronal excitability, neurotransmitter release, axon growth and long-term changes in gene expression, highlighting the central integrating position played by IP₃Rs and rendering them promising functional candidates in ASD pathophysiology.

In one embodiment of the present disclosure, the inventors have extended their observations from rare monogenic forms of autism to calcium signaling studies of primary, untransformed skin fibroblasts from patients with sporadic ASD. Significantly depressed Ca²⁺ release was observed in response to purinergic P2Y receptor activation in the fibroblasts from patients with typical sporadic ASD as well as monogenic ASD models.

### Example 24: Experiments and Methods

Materials:

Fluo-8 AM was purchased from AAT Bioquest, diluted in DMSO (Sigma D2650) to a stock concentration of 2 mM and frozen as 25 μl aliquots until needed. On the day of the experiment the Fluo-8 AM solution was thawed and diluted with an equal volume of 20% Pluronic F-127 (Molecular Probes, P6867) prepared in DMSO. Cal520 was purchased from AAT Bioquest, diluted in 20% pluronic F-127 solution in DMSO to a stock concentration of 1 mM and was frozen down into 2 μl aliquots until needed. The membrane permeant caged IP₃ analogue ci-IP₃/PM (D-2,3-O-Isopropylidene-6-O-(2-nitro-4,5-dimethoxy)benzyl-myo-Inositol 1,4,5-trisphosphate-Hexakis (propionoxymethyl) ester) was obtained from SiChem (Bremen, Germany), diluted in 20% pluronic F-127 solution in DMSO to a stock concentration of 200 μM and was frozen down into 2 μl aliquots until needed.

Enrolled Subjects with Typical ASD:

Subject enrollment into the Center for Autism Research and Translation (CART) involved a full day of testing, all obtained with informed consent and assent and complying with UCI IRB review. Subjects carrying a clinical diagnosis of ASD were enrolled. An age-appropriate research grade validated ADOS and IQ test were obtained, followed by a set of high-density EEG studies, a sleep deprivation study and preparation for a follow-up at home 5-day sleep study with accelerometers and app-assisted parent sleep and behavior logging. Metabolomic studies of blood, urine, saliva and volatile metabolites in breath were obtained.

Fibroblast Cells:

Primary, untransformed skin biopsy fibroblast cultures from neurotypical controls and monogenic forms of ASD (fragile X syndrome, tuberous sclerosis, Rett syndrome) were obtained from Coriell cell repository. Skin fibroblast cultures from CART-enrolled sporadic ASD subjects were established from skin biopsy explants. Only those subjects with validated ADOS scores in the “Autism” range were selected for study. All cells were cultured in Dulbecco's Modified Eagle's Media supplemented with 20% (v/v) fetal bovine serum without antibiotics at 37° C. in a humidified incubator gassed with 95% air and 5% CO₂, and used for up to 15 passages. Cells were studied at passages 10-15. For Ca²⁺ signaling studies, cells were detached with Ca²⁺, Mg²⁺-free 0.25% trypsin-EDTA (Life Technologies), harvested in normal growth media and sub-cultured on the 35 mm glass-bottom dishes for single cell studies, or on FLIPR plates for high-throughput studies, for 2 days to allow standard conditions prior to imaging studies.

High-Throughput Ca²⁺ Imaging.

Skin fibroblasts were seeded in clear-bottom black 96-well plates at 1×10⁴ cells per well and grown to confluency. On the day of the experiment, cells were loaded by incubation with 2 μM of the membrane-permeant Ca²⁺ indicator Fluo-8 AM in standard buffer solution (130 mM NaCl, 2 mM CaCl₂, 5 mM KCl, 10 mM glucose, 0.45 mM KH₂PO₄, 0.4 mM Na₂HPO₄, 8 mM MgSO₄, 4.2 mM NaHCO₃, 20 mM HEPES and 10 uM probenecid, pH 7.4 at the room temperature) with 0.1% fetal bovine serum for 1 h at 37° C., then washed with a Ca²⁺-free HBSS solution (120 mM NaCl, 4 mM KCl, 2 mM MgCl₂, 10 mM glucose, 10 mM HEPES, 1 mM EGTA, pH 7.4 at the room temperature). The solution was replaced with 100 μl of Ca²⁺-free HBSS solution in each well and cells were allowed to equilibrate for 5 minutes prior to assay with a Fluorometric Imaging Plate Reader (FLIPR; Molecular Devices, Sunnyvale, Calif.). A basal read of fluorescence in each well (470-495 nm excitation and 515-575 nm emission, expressed in arbitrary units; AU) was read for 2 seconds at 0.4 s exposure time. Next, 100 μl of 2×ATP (to 100 μM final concentration) or 100 μl of 2× ionomycin (to 1 μM final concentration) in Ca²⁺-free HBSS was added to a given well. Only a single recording was obtained from each well. Ionomycin-induced fluorescence changes from wells without prior addition of ATP were used to normalize ATP-evoked responses. Recordings were performed in triplicate. Each experiment was repeated on at least two independent days.

Single-Cell Ca²⁺ Imaging Studies:

Cells seeded in glass-bottomed dishes were loaded for imaging using membrane-permeant esters of Fluo-8 and caged i-IP₃ (ci-IP₃). Briefly, cells were incubated at room temperature in HEPES-buffered saline (2.5 mM CaCl₂, 120 mM NaCl, 4 mM KCl, 2 mM MgCl₂, 10 mM glucose, 10 mM HEPES) containing 1 uM ci-IP₃/PM for 45 mins, after which 4 uM Fluo-8 AM was added to the loading solution for further 45 minutes before washing three times with the saline solution. [Ca²⁺]ᵢ changes were imaged using a Nikon Eclipse microscope system with a 40× (NA=1.30) oil objective. Fluo-8 fluorescence was excited by 488 nm laser light, and emitted fluorescence (>510 nm) was imaged at 30 frames sec-1 using an electron-multiplied CCD Camera iXon DU897 (Andor). A single flash of UV (ultraviolet) light (350-400 nm) from an arc lamp focused to uniformly illuminate a region slightly larger than the imaging field was used to uncage i-IP₃, a metabolically stable isopropylidene analogue of IP₃, which evoked activity persisting for a few minutes. Image data were acquired as stack.nd2 files using Nikon Elements for offline analysis. Fluorescence signals are expressed as a ratio (ΔF/F₀) of changes in fluorescence (ΔF) relative to the mean resting fluorescence at the same region before stimulation (F₀). Recordings were performed in triplicate, and the measurement outcomes were compared using Mann-Whitney test.

Data Processing and Analysis:

The peak change in fluorescence amplitude (ΔF) in each well was normalized to the basal fluorescence of that well before stimulation (F₀) after subtraction of the camera black offset level. ATP responses were further normalized to the triplicate average response ΔF/F₀ of the ionomycin from each corresponding cell line from the same plate. Data represent mean values from triplicate wells±SEM. To mitigate plate-to-plate and day-to-day variability, mean responses for each cell line were divided by that of a reference cell line (GM03440) located in the top left corner of each plate. OriginPro 2015 was used for data analysis and graph plotting.

### Example 25: ATP Induced Ca²⁺ Signalings

ATP-Induced Ca²⁺ Signaling is Reduced in Patients with ASD.

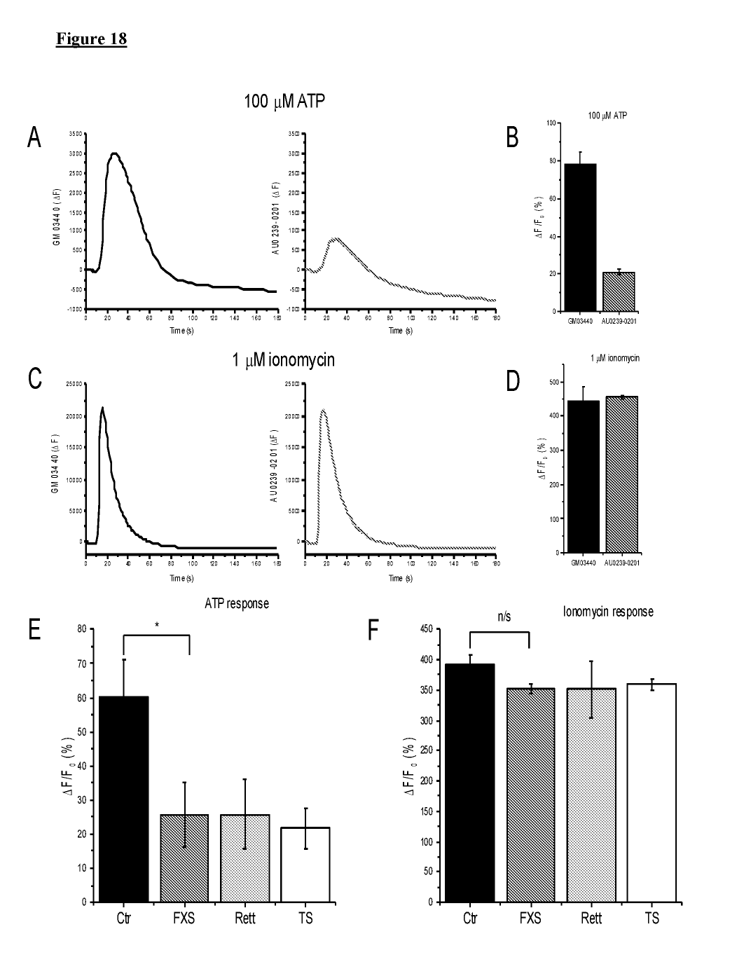

To measure intracellular Ca²⁺ release in patients with ASD, a high-throughput imaging system FLIPR (Fluorometric Imaging Plate Reader) was used. 100 μM ATP was applied to activate cell-surface purinergic GPCRs and induce subsequent IP₃ production and Ca²⁺ release via IP₃Rs in the absence of extracellular Ca²⁺ to avoid complications from Ca²⁺ entry across the cell membrane. Representative fluorescence traces of ATP responses from one control and one ASD cell line are shown on FIG. 18A, top. Fluorescence signals were quantified as a ratio (ΔF/F₀) of the fluorescence change (ΔF) at each well relative to the mean resting fluorescence (F₀) before stimulation. FIG. 18B demonstrates mean ΔF/F₀ values from these cell lines in response to 100 μM ATP run in triplicates (bar graphs represent mean values, error bars represent standard error mean of the triplicate recordings from three individual wells).

Amplitude of IP₃-mediated Ca²⁺ signaling strongly depends on the ER Ca²⁺ store filling, with larger store content resulting in greater Ca²⁺ release. To determine whether differences in ATP-evoked Ca²⁺ signals may arise from differences in intracellular Ca²⁺ store content, 1 μM ionomycin, a specific Ca²⁺ ionophore, were applied to independent wells (FIG. 18A, bottom). Ionomycin induces Ca²⁺ release from all intracellular organelles, thus it can serve as a measure of maximally available Ca²⁺ content. Peak ionomycin response amplitude normalized to the basal fluorescence (ΔF/F₀) from one ASD and one control subject was averaged from triplicate wells and presented as a mean±SEM (FIG. 18C). ASD and control cell lines had a similar Ca²⁺ response to ionomycin, consistent with results that maximal Ca²⁺ store filling does not differ between monogenic forms of ASD and unaffected subjects. To account for any differences between individual cell lines in the Ca²⁺ store filling across different 96-well plates and different days, all ATP-induced Ca²⁺ signals are presented as a ratio of the ATP response to the ionomycin response (FIG. 18D). To account for day-to-day variability typical for high-throughput screens such as FLIPR, each cell line's response was normalized to the same reference cell line that was plated on each of the 96 well plates during two individual runs, where all data were obtained. In one embodiment, IP₃-mediated Ca²⁺ release is reduced in monogenic models of ASD. In one embodiment, fibroblasts from patients with monogenic forms of ASD—fragile X, tuberous sclerosis and Rett syndromes—demonstrated significantly reduced ATP-mediated Ca²⁺ release. Despite providing great insight into pathophysiology of the disease, rare monogenic syndromes represented just a fraction of all ASD cases, with the majority being sporadic, or polygenic. To determine whether the IP₃-signaling defect is a common feature of ASD, or is inherently unique to single-gene mutations, these observations were expanded to fibroblasts from patients with sporadic forms of ASD. Sporadic ASD represented majority of all ASD cases and was thought to arise from a polygenic combination of inherited risk factors and environmental triggers, however, exact etiology of the process is largely unknown. Patients that were clinically diagnosed with autism or autism spectrum disorder were enrolled, and subjected primary fibroblasts cells derived from the patients to the same screening assay to assess how wide-spread this signaling defect would be. Primary skin fibroblast cultures were established from skin punch biopsy explants as previously described. Sporadic ASD cell lines demonstrated significantly depressed Ca²⁺ release in response to ATP in the absence of extracellular Ca²⁺, while the ionomycin response was not significantly different, suggesting defective IP₃-mediated Ca²⁺ release that did not result from different intracellular Ca²⁺ store filling, consistent with findings in monogenic models.

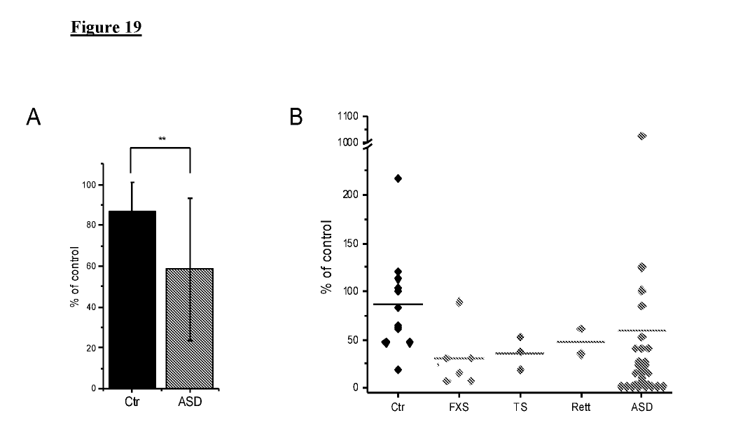

To assess significant statistical difference in the resulting data between sporadic ASD and control individuals, a receiver operating characteristic (ROC) curve was generated that was used for identifying parameters that are sensitive and specific enough to separate affected from unaffected individuals for diagnostic purposes. Each individual Ca²⁺ signaling value obtained from the screen can come from either an affected individual (true positive), or a healthy control (true negative), and be of a low amplitude, characteristic of ASD signaling pattern, or a high amplitude, more common for healthy individuals. After sorting all subjects by their Ca²⁺ signaling values, at very low value threshold, the inventors only had ASD subjects, while by increasing that threshold the inventors started including subjects without ASD. For each Ca²⁺ signaling test value threshold, there was a ratio of people who were disease positive (true positive) or disease negative (false positive). The goal of the test was to find a Ca²⁺ signaling value that would accurately discriminate between affected and unaffected individuals in majority of cases. High sensitivity implies that affected people were identified in most cases (i.e., few false negatives), and high specificity means that few unaffected individuals were identified as diseased (i.e., few false positives). Using Ca²⁺ signaling as a readout method, the inventors were able to achieve 83% sensitivity and 92% specificity for the test. The ROC curve was generated by plotting sensitivity (true positive rate) against specificity (false positive rate) at each test value. The area under the curve represented a useful tool to compare utility of different biomarkers. It showed the overall probability that the correct disease status (ASD vs unaffected) would be accurately identified in a randomly chosen patient, with the AUC of 1 having a perfect predictive value and 0.5 being of no diagnostic utility. The ROC generated from sporadic ASD patients resulted in a robust area under the curve (AUC) of 0.85 (FIG. 19), suggesting a good diagnostic value in predicting the disease status.

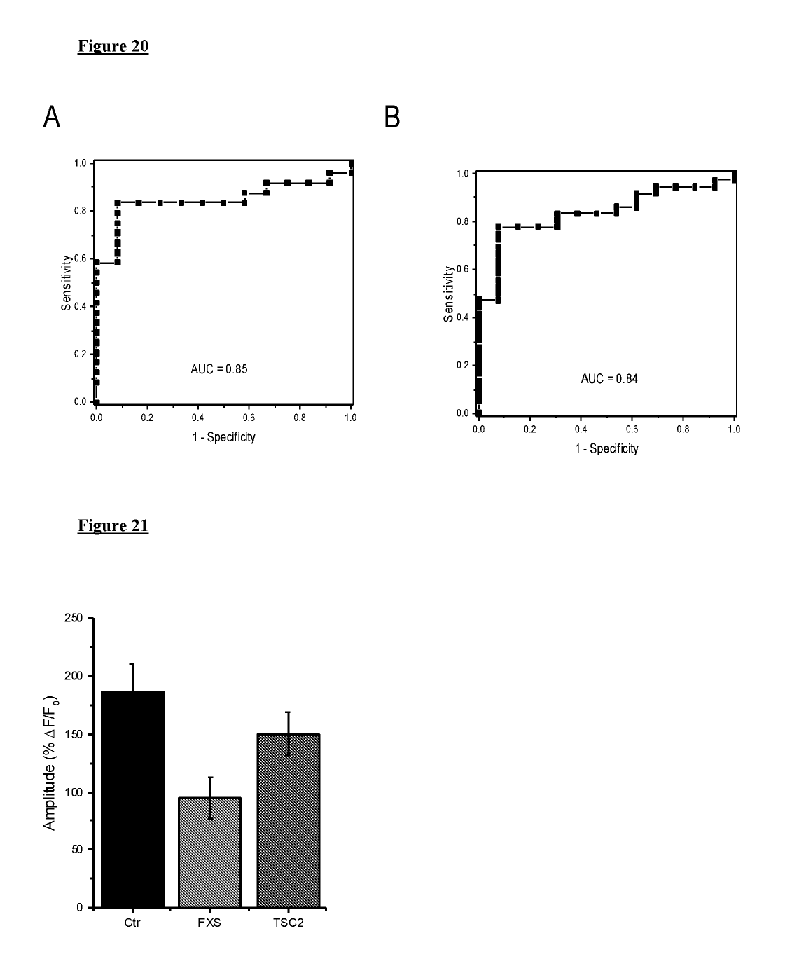

When the cohort of patients were expanded to include those with rare monogenic forms of ASD with known single gene mutations—fragile X, tuberous sclerosis and Rett syndromes—Ca²⁺ signaling values fall in the range of that of ASD patients (FIG. 20A). An ROC curve generated to include syndromic patients (FIG. 20B) yielded an AUC of 0.84 with 78% sensitivity with 92% specificity—remarkably similar to 0.85 AUC of sporadic autism only. These data suggested that the test does not discriminate between sporadic and syndromic forms, but more importantly, it also suggests a common underlying signaling deficit across different forms of ASD.

### Example 26: Ca²⁺ Signaling

In one embodiment, IP₃-mediated intracellular Ca²⁺ signaling is significantly affected in several monogenic models of ASD, and provide strong evidence that the same defect is present in patients with sporadic forms of ASD. Activation of purinergic P2Y signaling in primary human skin fibroblasts was inhibited in patients with sporadic ASD, and this defect was not dependent on intracellular Ca²⁺ store content. The experiments and results presented herein illustrate a strong connection between Ca²⁺ signaling and autism phenotype, independently of its genetic origin.

A high-throughput Ca²⁺ signaling screening assay was implemented on primary skin fibroblasts from autistic patients and derived an ROC curve with high sensitivity and specificity that could discriminate autism patients from unaffected controls to aid the diagnostic process. At present autism is diagnosed by clinicians based on a battery of behavioral tests that are possible only after the child starts displaying various behavior patterns—usually around the age of 2. The process of autism diagnosis may rely on questionnaires and observations from parents, teachers, and doctors—this makes the diagnosis subject to misinterpretation and human error. Autism diagnosis may also rely on the ADOS score, which is a scientific ASD diagnostic tool. In one embodiment, the inventors have presented a novel functional screening assay that identifies “at risk” individuals early in life and serves in combination with conventional diagnostic practices in autism. This test utilizes primary, non-transformed fibroblasts that could be easily obtained with a minimally-invasive biopsy, are easy to culture and maintain. Fibroblasts have proven useful in the research of several neurological conditions, including Alzheimer's and Huntington's diseases and as biomarkers for diagnostic purposes, much as is now routine in other organelle diseases, such as Tay-Sachs and Niemann-Pick diseases.

Intricate intracellular Ca²⁺ signaling arising from the ER is important for various cell functions related to cell survival and cell death. Recently Ca²⁺ signaling from the ER to mitochondria and lysosomes have been strongly implicated in induction and control of autophagy. Autophagy is a cellular process that directs degradation of proteins and even complete organelles, and under basal conditions it is necessary for cell survival and proliferation. By degrading misfolded and recycled proteins and old dysfunctional organelles autophagy provides protein quality control and assist in normal energy and cell homeostasis. Under nutrient deprivation autophagy maintains nutrient availability and ensures cell survival by catabolizing complex molecules into simpler building blocks. In the absence of constitutive IP₃-mediated Ca²⁺ signaling to mitochondria, autophagy is significantly upregulated in a number of cell types, suggesting a universal role of this type of signaling in cell survival and bioenergetics homeostasis.

### Example 27: IP3-Mediated Ca2+ Signaling

As disclosed herein, cytosolic Ca²⁺ homeostasis involves ion flux from intracellular organellar stores, as well as transport across the plasma membrane. Diseases of the intracellular organelles are an emerging area of medicine. In neurons, IP₃R-mediated Ca²⁺ release is involved in crucial functions-including synaptic plasticity and memory, neuronal excitability, neurotransmitter release, axon growth and long-term changes in gene expression-highlighting the central integrating position played by IP₃Rs. Ca²⁺ release is activated in response to the second messenger inositol 1,4,5-trisphosphate (IP₃), which is produced upon stimulation of G_{q} protein-coupled (GPCRs) and tyrosine kinase-linked cell surface receptors. The specificity of the resulting cellular responses is ensured by an exquisite temporo-spatial patterning of cytosolic Ca²⁺ signals. Opening of the IP₃R channel requires not only IP₃, but also binding of Ca²⁺ to receptor sites on the cytosolic face. This leads to biphasic regulation, such that small elevations of cytosolic Ca²⁺ induce channel opening, whereas larger elevations cause inactivation. The positive feedback by Ca²⁺ (Ca²⁺-induced Ca²⁺ release; CICR), may remain restricted to individual or clustered IP₃Rs, producing local Ca²⁺ signals known, respectively, as Ca²⁺ blips and puffs, or may propagate throughout the cell as a saltatory wave by successive cycles of Ca²⁺ diffusion and CICR. Thus, IP₃-mediated Ca²⁺ signaling represents a hierarchy of Ca²⁺ events of differing magnitudes. The spatial patterning it orchestrates is critical to proper cellular function, and disruptions in the magnitude and organization of neuronal Ca²⁺ signals may contribute to the pathogenesis of ASD.

In accordance with embodiments herein, disclosed herein are use of primary, untransformed skin fibroblasts derived from patients with FXS and TS to evaluate ASD-associated functional deficits in IP₃-mediated Ca²⁺ signaling. Identification of disease-specific signaling defects in skin cells can be used as biomarkers for diagnostic purposes, much as is now routine in other organelle diseases, such as Tay-Sachs and Niemann-Pick diseases, and through which novel therapies for these diseases have emerged. The results and disclosure herein demonstrate that IP₃-mediated Ca²⁺ signals are significantly depressed in fibroblasts from both FXS and TS patients and, by resolving signals at the single-channel level, fundamental defects in IP₃R channel activity in ASD. Thus, dysregulated IP₃R signaling is a nexus where genes altered in ASD converge to exert their deleterious effect.

In accordance with embodiments herein, disclosed herein are abnormalities of IP₃-mediated Ca²⁺ signaling in three distinct genetic models that display high co-morbidity with ASD—fragile X syndrome and two genetically-distinct forms of tuberous sclerosis (TSC1 and TSC2). Ca²⁺ responses evoked by agonist stimulation of GPCR-mediated IP₃ signaling were significantly smaller in fibroblasts derived from patients with FXS and TS, as compared with matched control cell lines. In contrast, no significant differences were found in Ca²⁺ liberation evoked by application of the Ca²⁺ ionophore, ionomycin. This indicated that the diminished responses to IP₃ did not result from diminished ER Ca²⁺ store content. Moreover, Ca²⁺ signals evoked by intracellular uncaging of IP₃ were depressed in FXS and TS cell lines, pointing to a deficit at the level of Ca²⁺ liberation through IP₃Rs and not solely because of diminished GPCR-mediated production of IP₃. The depression of Ca²⁺ signals cannot be attributed entirely or substantially to reduced expression of IP₃R proteins, because mean agonist-evoked Ca²⁺ responses across four FXS and TS lines were about 22% of matched controls, whereas western blots showed mean IP₃R levels to be about 80% of controls and uncorrelated with the extent of Ca²⁺ signaling depression in these different cell lines.

In accordance with embodiments herein, by resolving Ca²⁺ liberation during ‘elementary’, local signals evoked by photoreleased IP₃, the inventors demonstrate that defects in global Ca²⁺ signaling in these three distinct ASD-associated models are reflected at the level of Ca²⁺ release through individual and small clusters of IP₃Rs. In both FXS and TS cell lines, fewer sites of local Ca²⁺ release were observed compared to a control cell line, and the durations of these events were shorter. Because functional sites are comprised of clusters of small numbers of individual IP₃Rs, the amplitude of the fluorescence signal at a site depends on the channel permeability, together with the number of active channels in the cluster. Similar amplitudes of local Ca²⁺ signals across the cell lines was observed, suggesting that the Ca²⁺-permeation properties and cluster organization of IP₃Rs are not appreciably affected in FXS and TS. However, the shorter average duration of local events points to a modulation of IP₃R gating kinetics, and would lead to an overall decrease in amount of Ca²⁺ released over time. Compounding this, the numbers of local Ca²⁺ release sites within a cell was dramatically lower in FXS and TS cells as compared with control cells (respectively, 87% and 70%), although it is possible that the short duration events observed in the mutants may have contributed to undercounting their release sites. Taken together, these findings on local IP₃-mediated Ca²⁺signals illustrate that the deleterious effects of FXS and TS mutations are manifest at the level of the functional channel gating of IP₃Rs.

In accordance with embodiments herein, the IP₃R is a key signaling hub in the canonical metabotropic glutamate receptor (mGluR) pathway in neurons, and the mGluR theory of FXS postulates that disrupted mGluR signaling underlies the pathogenesis of the disorder. Activation of mGluRs leads to a brief hyperpolarization followed by a more prolonged depolarization. The initial outward current results from the opening of small conductance Ca²⁺-activated K⁺ channels. This current is proportional to the Ca²⁺ signal amplitude; and can be triggered directly by intracellular uncaging of IP₃. As a result, IP₃-evoked Ca²⁺ release transiently hyperpolarizes the cell and briefly depresses neuronal excitability, leading to a reduction in firing frequency. Suppressed IP₃-mediated Ca²⁺ release from the internal stores, as the inventors report in diverse models of ASD, is thus expected to diminish the inhibitory K⁺ conductance. This would tend to produce neuronal hyperexcitability, consistent with observations following mGluR stimulation of ASD-model neurons. A complex array of downstream signals arises from mGluR activation-whereas IP₃R Ca²⁺ signaling is one immediate downstream target, its function has not yet been molecularly dissected in ASD. At present, there is no direct extrapolation of the present results to IP₃ mediated signaling in neurons, given that fibroblasts predominantly express type 3 IP₃Rs whereas neurons predominantly express type 1 IP₃Rs. Nevertheless, because expression levels of all three isotypes of IP₃Rs are only slightly diminished in FXS and TS fibroblasts, the pronounced depression of Ca²⁺ signaling does not result from diminished expression of a specific isotype. Instead, the depressed Ca²⁺ signals likely result from modulatory effects on IP₃R function, which might extend across different isotypes.

Depression of IP₃-mediated Ca²⁺ signaling may further disrupt neurodevelopment through separate mechanisms. IP₃Rs have been shown to be central participants in autophagy. Decreased levels of autophagy result in defective synaptic pruning, which has been repeatedly associated with ASD in humans and mouse models, and promotion of autophagy rescues behavioral defects in mouse models of ASD.

Because of the ubiquitous nature of IP₃R signaling and its diverse roles in almost all cells of the body, deficits in IP₃-mediated Ca²⁺ signaling may not be limited to neurological correlates of ASD, but may also explain other characteristic ASD-associated heterogeneous symptoms, such as those of the gastrointestinal tract and immune system. Furthermore, since the ER serves as a sensor of a host of environmental stressors, this same mechanism may contribute to the known environmental component to the ASD phenotype, and holds the potential to reveal relevant stressors.

In accordance with embodiments herein, the present disclosure illustrates that ER IP₃R signaling is affected in three distinct genetic models of ASD, pointing to the ER as a functional “hub” where different cellular signaling pathways merge to contribute to the pathogenesis of ASD. In addition to its role in Ca²⁺ homeostasis, the ER serves as a key integrator of environmental stressors with metabolism and gene expression, as it mediates a host of broad ranging cell stress responses such as the heat shock and unfolded protein responses. In this light it can be seen to integrate a matrix of ASD associated risk factors. The IP₃R is identified as a functional target in monogenic models of ASD. Ca²⁺ screening in skin fibroblasts, which are routinely acquired as clinical specimens, may thus offer a promising technique in conjunction with behavioral testing for early detection of ASD, and for high-throughput screening of novel therapeutic agents.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the selection of constituent modules for the inventive compositions, and the diseases and other clinical conditions that may be diagnosed, prognosed or treated therewith. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

What is claimed is:

1. A kit for determining susceptibility to Autism Spectrum Disorder (ASD) in a subject, comprising:
an assay for determining an increase or decrease of IP₃R Ca²⁺ signaling activity levels in a cell,
wherein a decrease of IP₃R Ca²⁺ signaling activity is indicative of ASD susceptibility in the subject.

2. The kit of claim 1, wherein IP3R Ca²⁺ signaling activity is determined using one or more biomarkers that is a clinically tractable discriminant of ASD.

3. The kit of claim 1, wherein the ASD is monogenic ASD and/or sporadic ASD.

4. The kit of claim 1, wherein the monogenic form of ASD comprises FXS, TSC1, and/or TSC2.

5. The kit of claim 1, wherein the cell is a fibroblast cell.

6. The kit of claim 1, wherein the cell is a neuronal cell.

7. The kit of claim 1, wherein the decrease of IP3R Ca²⁺ signaling activity arises at the IP3R channel without a mutation in IP3R.

8. The kit of claim 1, wherein IP3R Ca²⁺ signaling activity is measured by imaging Ca²⁺ flux though single ion channels within intact cells with single channel resolution.

9. The kit of claim 1, wherein IP3R Ca²⁺ signaling activity is measured by the following:
a. using total internal reflection microscopy together with a slow Ca²⁺ buffer to restrict excitation of a cytosolic fluorescent Ca²⁺ indicator to within 100 nm of the plasma membrane;
b. monitoring the local microdomain of elevated cytosolic [Ca²⁺] around the pore of Ca²⁺-permeable membrane channels; and
c. dissecting the Ca²⁺ puffs arising from clusters of IP₃Rs by using localized single-channel Ca²⁺ fluorescence transients.

10. The kit of claim 1, wherein a change in the Ca²⁺ signaling activity is determined through changes in the spatial distribution of IP₃R channels as imaged by super-resolution imaging.

11. The kit of claim 1, wherein IP3R Ca²⁺ signaling activity is determined by the following:
a. monitoring cytosolic Ca²⁺ signals in skin fibroblasts from FXS and matched control subjects using a fluorimetric imaging plate reader;
b. applying ATP to activate GPCR-linked purinergic P2Y receptors in Ca²⁺ free extracellular solution to exclude Ca²⁺ influx through plasmalemmel channels; and
c. determining changes in IP3R Ca²⁺ signaling activity.

12. The kit of claim 1, wherein IP3R Ca²⁺ signaling activity is determined by the following:
a. obtaining equivalent amounts of separately cultured skin fibroblast cells from the patient and from the control individual, wherein the cultured skin fibroblast cells from each of the patient and the control individual have been loaded with a Ca²⁺ fluorescent probe, and contacted with an agonist of IP3R Ca²⁺ signaling;
b. measuring, in each of the cultured skin fibroblast cells from the patient and the individual obtained in (a), an amount of fluorescence emitted by the Ca²⁺ fluorescent probe; and
c. comparing the amounts of emitted fluorescence measured in (b).

13. A method of screening for a therapeutic agent for Autism Spectrum Disorder (ASD), comprising:
a. providing a cell sample of a subject diagnosed with ASD;
b. assaying for IP3R Ca²⁺ signaling activity in the cell sample in the presence of a candidate drug;
c. assaying for IP3R Ca²⁺ signaling activity in the cell sample in the absence of the candidate drug; and
d. determining that the candidate drug is a suitable therapeutic agent for treatment of ASD if the IP3R Ca²⁺ signaling activity is higher in the presence of the candidate drug than in its absence.

14. The method of claim 13, wherein the ASD is monogenic ASD and/or sporadic ASD.

15. The method of claim 14, wherein the monogenic form of ASD comprises FXS, TSC1, and/or TSC2.

16. The method of claim 13, wherein the cell sample comprises a skin fibroblast cell sample, an amniocyte cell sample obtained prenatally by amniocentesis, and/or a neuronal cell sample.

17. The method of claim 13, wherein the IP3R Ca²⁺ signaling activity is at the IP3R channel and without a mutation in the IP3R.

18. The method of claim 13, wherein IP3R Ca²⁺ signaling activity is measured by imaging Ca²⁺ flux though single ion channels within intact cells with single channel resolution.

19. The method of claim 13, wherein IP3R Ca²⁺ signaling activity is measured by the following:
a. using total internal reflection microscopy together with a slow Ca²⁺ buffer to restrict excitation of a cytosolic fluorescent Ca²⁺ indicator to within 100 nm of the plasma membrane;
b. monitoring the local microdomain of elevated cytosolic [Ca²⁺] around the pore of Ca²⁺-permeable membrane channels; and
c. dissecting the Ca²⁺ puffs arising from clusters of IP₃Rs by using localized single-channel Ca²⁺ fluorescence transients, wherein the single-channel Ca²⁺fluorescence transients turn on and off rapidly, tracking channel openings and closings with a time resolution of a few milliseconds.

20. The method of claim 13, wherein a change in the Ca²⁺ signaling activity is determined through changes in the spatial distribution of IP3R channels as imaged by super-resolution imaging.

21. The method of claim 13, wherein IP₃R Ca²⁺ signaling activity is determined by an assay comprising:
a. monitoring cytosolic Ca²⁺ signals in skin fibroblasts from FXS and matched control subjects using a fluorimetric imaging plate reader;
b. applying ATP to activate GPCR-linked purinergic P2Y receptors in Ca²⁺ free extracellular solution to exclude Ca²⁺ influx through plasmalemmel channels; and
c. determining changes in IP3R Ca²⁺ signaling activity.

22. A method for diagnosing susceptibility of autism spectrum disorder (ASD) in a subject, comprising:
a. obtaining a sample from the subject;
b. assaying the sample to determine IP3R Ca²⁺ signaling activity levels; and
c. comparing said signal activity level to a reference value based on the IP3R Ca²⁺ signaling activity in a similar sample from a healthy control subject;
wherein a lower activity level than the reference value in the sample is indicative of ASD.

23. The method of claim 22, further comprising administering an ASD treatment to the subject.

24. The method of claim 23, wherein the ASD treatment comprises a therapeutically effective dosage of a composition comprising one or more agonists of inositol triphosphate receptor (IP3R) calcium (Ca²⁺) signaling.

25. The method of claim 22, wherein reduction of IP3R Ca²⁺ signaling activity disrupts the normal mitochondrial bioenergetics, creating the energy deficient endophenotype of ASD.

26. A method of diagnosing Autism Spectrum Disorder (ASD) in a subject, comprising:
a. obtaining a sample from the subject;
b. activating one or more purinergic receptors in a cell sample of the subject;
c. measuring IP₃-mediated Ca²⁺ release in the cell sample; and
d. diagnosing ASD in the subject if IP₃-mediated Ca²⁺ release is depressed compared to a healthy control subject without ASD.

27. The method of claim 26, wherein the ASD is a syndromic and/or a sporadic form.

28. The method of claim 26, wherein the depressed level of Ca²⁺ release is not due to different endoplasmic reticulum Ca2+ content.

29. The method of claim 28, wherein different endoplasmic reticulum Ca2+ content is judged by response to one or more Ca²⁺ ionophores.

30. The method of claim 26, wherein the IP₃-mediated Ca²⁺ release is from an endoplasmic reticulum.
